(19) United States
(12) Patent Application Publication
(10) Pub. No.: US 2026/0241146 A1
(43) Pub. Date: Aug. 20, 2026

(54) MEDICAL DEVICES FOR FLUID DELIVERY AND METHODS OF USE AND MANUFACTURE

(71) Applicant: Encompass Vascular, Inc., San Jose, CA (US)

(72) Inventors: Jean C. ORTH, Morgan Hill, CA (US); Zaya TUN, Livermore, CA (US); Robert G. QUINTOS, Newark, CA (US); Vu A. LAM, San Jose, CA (US)

(21) Appl. No.: 19/157,665

(22) PCT Filed: Aug. 8, 2023
(86) PCT No.: PCT/US2023/071870

§ 371 Date: Aug. 18, 2025

Related U.S. Application Data
(60) Provisional application No. 63/486,234, filed on Feb. 21, 2023.
(60) Provisional application No. 63/502,886, filed on May 17, 2023.
(60) Provisional application No. 63/513,219, filed on Jul. 12, 2023.

(51) Int. Cl. A61M 25/10; A61B 18/00; A61B 18/06

(52) U.S. Cl. CPC A61M 25/10; A61B 2018/00434; A61B 2018/00511; A61B 2018/00577; A61B 18/06; A61M 2025/105

(57) ABSTRACT

Medical devices and methods for delivering fluid for renal denervation. The medical devices include one or more needles for delivering fluid at different depths. The methods may optionally include expanding an expandable member such as an inflatable member to expand an expandable scaffold outward toward a renal artery wall.

### CROSS REFERENCE TO RELATED APPLICATIONS

This application claims priority to the following U.S. Provisional Applications: 63/486,234, filed Feb. 21, 2023; 63/502,886, filed May 17, 2023; and 63/513,219, filed Jul. 12, 2023, the entire disclosures of which are incorporated by reference herein for all purposes.

This application incorporates by reference herein for all purposes the entire disclosures of U.S. Pat. No. 11,071,847, issued Jul. 27, 2021; WO2021/133966, published Jul. 1, 2021; WO2022/182598, published Sep. 1, 2022; PCT/US2023/069886, filed Jul. 10, 2023; and WO2022/232589, published Nov. 3, 2022.

All publications and patent applications mentioned in this specification are herein incorporated by reference to the same extent as if each individual publication or patent application was specifically and individually indicated to be incorporated by reference.

### FIELD

Intravascular (e.g., perivascular or adventitial) delivery of agents. In some embodiments, the methods can be used for the treatment of peripheral vascular disease, inclusive of both peripheral artery disease and peripheral venous disease. In some embodiments, the methods and devices are adapted to ablate nerves disposed within and outside of the vessel wall to disrupt neural communication along the nerves, such as renal denervation (“RDN”) for the treatment of uncontrolled hypertension (HTN) and targeted lung denervation for the treatment of chronic obstructive pulmonary disease (COPD).

### BACKGROUND

For the treatment of HTN, several attempts have been made to ablate nerves disposed within and outside the renal arteries to disrupt the neural communication along those nerves, generally referred to herein as renal denervation (“RDN”). Previous understanding of renal nerve distribution was that the vast majority of nerves are located within 3 mm of the vessel lumen. New understanding shows proximal distribution of the nerves extend much deeper, with dense concentration of nerves located up to 8 mm in depth from the vessel lumen.

Approaches are needed that can ablate nerves situated as far as 8 mm from the renal artery to provide a more comprehensive ablation of the nerves. Approaches may also be needed that can deliver an ablative agent to different distances from the arterial wall at different axial locations along the renal artery to more effectively address the change in location and concentration of nerves at the different axial locations along the renal artery.

### SUMMARY OF THE DISCLOSURE

This disclosure includes some aspects that are related, but is not limited to, intravascular delivery of neurolytic agents for the purpose of chemical renal denervation. While examples in this disclosure may refer to delivery of agents for peripheral vascular disease (arterial or venous), it is understood that some of the concepts, features, and methods herein may be applicable in the context of RDN, and vice versa.

One aspect of the disclosure is an intravascular apparatus adapted for delivery of a fluid for renal denervation.

In this aspect, the apparatus may comprise an inflatable balloon, optionally having an inflated cylindrical configuration.

In this aspect, the apparatus may comprise an expandable infusion scaffold comprising one or more spines disposed about an outer cylindrical surface of the inflatable balloon. Any of the spines herein may also be referred to as infusion spines.

In this aspect, the one or more spines may comprise a plurality of radial openings therethrough, each of the plurality of radial openings associated with a needle that has a delivery configuration within a spine lumen of the spine in which a distal tip of the needle is radially constrained by the spine, and a deployed configuration in which the needle extends radially outward from the associated opening after the needle is moved axially (pushed distally or retracted proximally) relative to the spine to facilitate delivery of an agent from the needle.

In this aspect, the inflatable balloon may have a tapered proximal end and a tapered distal end, and wherein the cylindrical configuration of the balloon is in between the tapered proximal and distal ends. One or more spines may also be disposed about the optionally tapered proximal end.

In this aspect, a plurality of needles associated with a spine may be adapted to be axially moved together (distally or proximally) as a unit relative to the associated spine, and may optionally be secured to a rail that is disposed within the spine.

In this aspect, one or more spines may be arranged helically about the cylindrical surface of the inflatable balloon.

In this aspect, one or more spines may be arranged axially about the cylindrical surface of the inflatable balloon.

In this aspect, a plurality of needles associated with each of the one or more spines may be operatively coupled such that they are adapted to be moved axially as a group relative to the associated spine, optionally coupled to a rail within the spine. The optional rail may comprise a rail lumen that is in fluid communication with the plurality of needles.

In this aspect, each of the plurality of needles may be in fluid communication with a distinct fluid delivery lumen, which may optionally be in fluid communication with a rail lumen.

In this aspect, one or more spines may extend along at least half of the length of the portion of the balloon that has the inflated cylindrical configuration.

In this aspect, a portion of the balloon that has the inflated cylindrical configuration may have a length from 10 mm to 40 mm, optionally from 10 mm-30 mm.

In this aspect, the expandable infusion scaffold may be attached to the inflatable balloon along at least a portion of a length of the scaffold.

In this aspect, the expandable infusion scaffold may not be attached to the inflatable balloon.

In this aspect, one or more spines may each have a stiffness that is not constant along the length of the inflatable member.

In this aspect, a plurality of needles may be operatively coupled to an axially moveable rail that is disposed within the associated spine lumen, and wherein the rail may have a stiffness that is not constant along the inflatable balloon.

In this aspect, one or more spines may comprise one or more of nitinol, stainless steel, polymer, polyimide, or a braided member.

One aspect of the disclosure is an intravascular apparatus adapted for delivery of a fluid. In this aspect, the apparatus may include an inflatable balloon having an inflated cylindrical configuration and an expandable infusion scaffold.

In this aspect, the expandable infusion scaffold may comprise one or more spines, any of which may be disposed about an outer cylindrical surface of the inflatable balloon.

One aspect of this disclosure is a method of intravascular fluid delivery, for diagnostic purposes and/or treatment.

In this aspect, the method may include advancing an intravascular apparatus to a target location within a vessel; inflating a balloon toward a cylindrical configuration to cause one or more spines of an expandable infusion scaffold to expand toward a vessel wall and be disposed about an outer cylindrical surface of the balloon; moving a plurality of needles axially within the one or more spines and deploying the plurality of needles out of radial openings of the spines such that tips of each of the plurality of needles pierce into the vessel wall; and delivering a fluid agent out of the plurality of needles and into the vessel wall.

Delivering a fluid agent out of the plurality of needles and into the vessel wall may comprise delivering the fluid agent into at least one of the media, adventitia, perivascular tissue of the vessel wall, and outside of the perivascular space. In this aspect, delivering a fluid agent may comprise delivering a neurolytic agent out of the plurality of needles and into and/or outside of the vessel wall to ablate renal nerves.

One aspect of the disclosure is a method of denervating renal nerves, comprising advancing an intravascular ablation apparatus into a renal artery; delivering an ablative agent into tissue along a renal artery lumen at a first depth at a first axial location of the renal artery to ablate renal nerves along the renal artery; and delivering the ablative agent into tissue along the renal artery to a second depth at a second axial location of the renal artery to ablate the same and/or different nerves along the renal artery, the second axial location being distal to the first axial location, and the first depth being greater than the second depth. In this aspect, the two delivering steps may occur simultaneously, and wherein the first delivering step may be performed with a proximal plurality of needles, and the second delivering step may occur with a distal plurality of needles, the proximal plurality of needles, when in deployed configurations, extend further radially outward than the distal plurality of needles in deployed configurations.

In one aspect of the disclosure, any of the apparatuses herein may include an expandable infusion scaffold that comprises a spine disposed about an outer surface of the inflatable balloon. The spine may include a proximal needle that extends further radially outward than a distal needle when the two needles are in their deployed configurations. The apparatus may include one or more of any of the features herein.

One aspect of the disclosure is a needle that comprises a visualization marker, such as a radiopaque marker band (e.g., as shown in FIGS. 20, 21A and 21B). The needle(s) optionally comprises a non-deployable portion (such as shown in FIG. 20) and a deployable portion (such as shown deployed in FIG. 20), wherein the non-deployable portion comprises the visualization marker (such as shown in FIGS. 20, 21a and 21b). Any of the apparatuses herein may include a plurality of needles, wherein less than all of the needles associated with a rail within a given spine comprise a visualization marker, and optionally wherein only distal needles in the spines comprise a visualization marker.

One aspect of the disclosure is an inflatable balloon that comprises a first material and a second material that is different than the first material. The first material is optionally an inner balloon layer, and the second material is optionally an outer balloon layer that is secured to the inner balloon layer (directly or indirectly). The first material is optionally nylon and the second material is optionally polyurethane.

In this aspect, any of the spine sleeves herein (including material incorporated by reference) that are bonded to the inflatable member (wherein the sleeve houses a spine therein), may be bonded to an outer balloon layer comprising the second material of the inflatable member. Sleeve(s) and the second material (of the outer balloon layer) may optionally be the same material, optionally polyurethane. The sleeve(s) may optionally comprise a PEBAX® material. In this aspect, with designs in which any of the spine sleeves herein that are bonded to an inflatable member (wherein the sleeve houses a spine therein), the material of the sleeve(s) and the material of a balloon outer layer are optionally better adapted to form a stronger adhesive or thermal bond than the material of the sleeve(s) and an inner layer of the balloon.

One aspect of the disclosure is related to an agent delivery device and methods of delivering one or more agents.

In this aspect, the device may include an inflatable balloon having an inflated configuration, optionally cylindrical when inflated.

In this aspect, the device includes one or more expandable infusion spines, each of the one or more infusion spines defining a lumen therein and each including a plurality of spaced apart radial openings through an optionally curved outer surface of the infusion spine. Expandable infusion spines may be adapted and positioned about an inflatable balloon such that inflation of the balloon causes the scaffold to radially expand, the one or more infusion spines extending about an outer surface of the inflatable balloon (optionally a cylindrical surface) when the inflatable balloon is in an inflated state.

In this aspect, the device may include a plurality of movable needle assemblies, each one of the plurality of movable needle assemblies disposed in one of the one or more expandable infusion spines and movable within and relative to the infusion spine.

In this aspect, the devices may include an elongate multi-lumen comprising a plurality of lumens, wherein a portion of each of the one or more infusion spines is disposed within a spine lumen of the multi-lumen.

In this aspect, a proximal end region of a balloon is optionally adhered to and disposed within a distal region of a balloon inflation lumen of the multi-lumen. A guidewire lumen optionally extends through the balloon inflation lumen of the multi-lumen, and wherein the guidewire lumen extends through at least a portion of the catheter shaft and through the length of the balloon.

In this aspect, at least a portion of the one or more spines optionally extend along an entire length of the multi-lumen.

In this aspect, each of the one or more spines optionally comprise at least two different materials in first and second sections along the length of the spine, and wherein the spine comprises a first material (e.g., stainless steel) in a first section, and a second material (e.g., PEEK) in a second section within a spine lumen of the multi-lumen. A proximal end of a first section and a distal end of a second section are optionally coupled together at a coupling location within the spine lumen of the multi-lumen.

In this aspect, one or more spines optionally do not extend along an entire length of the multi-lumen. Each of the one or more spines optionally has a proximal end that is disposed within a distal end region of one of the spine lumens of the multi-lumen. Any of the spine lumens of the multi-lumen optionally functions as a second section of the respective spine, providing a continuous conduit through which each of the plurality of movable assemblies is advanced along the length of the multi-lumen, or functions as a second section of a secondary spine.

In this aspect, the multi-lumen optionally comprises from one to ten spine lumens, optionally three spine lumens.

In this aspect, the multi-lumen may comprise a balloon inflation lumen. A guidewire lumen mat extend through the balloon inflation lumen.

In this aspect, the multi-lumen optionally comprises a separate guidewire lumen.

In this aspect, in a sectional view, the spine lumens are optionally disposed more on a first side of the multi-lumen than a second side of the multi-lumen.

In this aspect, the multi-lumen optionally comprises one or more primary spine lumens and one or more secondary spine lumens, which optionally have the same or different diameters.

In this aspect, the multi-lumen optionally comprises a first set of primary spine lumens with a first diameter, and a second set of secondary spine lumens with a second diameter that is less than the first diameter. One or more primary spines are optionally each disposed in a primary spine lumen of the multi-lumen, and one or more secondary spines are optionally each disposed in a secondary spine lumen of the multi-lumen. More than one secondary spine is optionally disposed in a single secondary spine lumen of the multi-lumen, optionally wherein three secondary spines are disposed in a single secondary spine lumen of the multi-lumen.

In this aspect, each of the plurality of movable assemblies optionally include a rail that includes a rail lumen, a plurality of needles, and one or more fluid delivery lumens within the rail lumen that are in fluid communication with the plurality of needles, and wherein the plurality of needles are coupled to the rail such that movement of the rail within the corresponding infusion spine moves the plurality of the needles as a group relative to the infusion spine between undeployed configurations in which each of the plurality of needles are housed within the infusion spine and deployed configurations in which each of the plurality of needles extends generally radially out of one of the radial openings in the infusion spine for delivery of an agent into a wall of a target vessel.

One aspect of the disclosure is an agent delivery device, comprising one or more expandable infusion spines, each of the one or more infusion spines defining a lumen therein and each including a plurality of spaced apart radial openings through an optionally curved outer surface of the infusion spines, wherein the one or more infusion spines each comprise a first section with a first material, and a second section with a second material different than the first material. Any other feature of any other device or apparatus herein may be included in this aspect.

In this aspect, the first and second sections are optionally coupled together (directly or indirectly) within a catheter shaft that is proximal to the balloon, and wherein the catheter shaft is optionally a multi-lumen. The catheter shaft may comprise a shaft lumen, and wherein two or more infusion spines are optionally positioned in the shaft lumen.

In this aspect, the first material may comprise stainless steel (and is optionally only stainless steel), and the second material optionally comprises PEEK (and is optionally only PEEK).

In this aspect, the first section is optionally distal to the second section, and a proximal end of the first section is optionally positioned in a distal end of a lumen, optionally the distal end of a spine lumen in a multi-lumen.

In this aspect, the one or more infusion spines optionally extend along an entire length of a catheter shaft. The catheter shaft is optionally a multi-lumen. The catheter shaft optionally comprises a shaft lumen, and wherein two or more infusion spines are optionally positioned in the shaft lumen.

### BRIEF DESCRIPTION OF THE DRAWINGS

FIG. 1 is a side view of a distal region of an exemplary infusion device including an expandable scaffold in an expanded configuration.

FIG. 2A is a side view of a distal region of an exemplary infusion device including an expandable scaffold in an expanded configuration.

FIG. 2B is a side view of a distal region of an exemplary infusion device from FIG. 2A with needles deployed from elongate spines of the scaffold.

FIG. 3A is an end view of a distal region of an exemplary infusion device with an inflatable member inflated.

FIG. 3B is an end view of a distal region of the exemplary infusion device in FIG. 3A, shown with needles deployed.

FIG. 4A is an end view of a distal region of the exemplary infusion device from FIG. 3A, shown within an exemplary vessel.

FIG. 4B is an end view of a distal region of the exemplary infusion device in FIG. 3A shown with needles deployed and within an exemplary vessel.

FIG. 5 is a distal region of an exemplary infusion device illustrating needles deployed from spines of an expandable scaffold.

FIGS. 6A-6D illustrate views of portions of an exemplary needle sub-assembly or rail track sub-assembly.

FIG. 6E illustrates an exemplary needle secured to a fluid delivery lumen.

FIG. 6F illustrates an exemplary rail.

FIG. 6G illustrates a portion of an exemplary infusion spine.

FIG. 7A illustrates a top view of an exemplary needle or rail track sub-assembly.

FIG. 7B illustrates a side view of the exemplary needle or rail track sub-assembly from FIG. 7A.

FIG. 8 is a side view of a plurality of exemplary needles deployed outward from an infusion spine.

FIG. 9 illustrates an exemplary cross section of an exemplary needle or rail track sub-assembly.

FIG. 10 illustrates an exemplary cross section of an exemplary needle or rail track sub-assembly.

FIG. 11A illustrates a side view of an exemplary infusion device in a lower profile configuration.

FIGS. 11B and 11C illustrate side and end views, respectively, of the exemplary infusion device from FIG. 11A in an expanded configuration with needles deployed.

FIG. 12 illustrates a distal region of an exemplary infusion device in an expanded configuration, with regions that are more flexible than other sections of the spine.

FIG. 13 is a side view illustrating an exemplary infusion device, including a proximal region positioned to be disposed outside of a patient.

FIG. 14 is a side view of an exemplary proximal region of an exemplary infusion device, including an exemplary actuator.

FIGS. 15A and 15B are proximal end views of a proximal external region of an exemplary infusion device.

FIG. 16 is a side view illustrating an exemplary manner in which an inflatable member may be secured to a catheter shaft.

FIG. 17a is a representation of the renal artery and surrounding nerves.

FIGS. 17b and 17c illustrate, at different axial locations along a renal artery shown in FIG. 17a, the renal nerve distribution stratified according to the total number and relative number of nerves.

FIG. 17c′ is a close-up view of the section from FIG. 17c, including in better detail radial distances from the renal artery lumen.

FIG. 18 shows a portion of an ablation device adapted for renal denervation, including a first set of needles extending further radially than a second set of needles when in their relative deployed configurations.

FIG. 19 illustrates an exemplary total tissue ablation region, including a proximal ablation zone and a distal ablation zone, the proximal ablation zone extending further radially outward than the distal ablation zone. The tissue ablation region (as well as the ablation zones) extends at least partially circumferentially relative to the renal artery lumen.

FIG. 20 illustrates an exemplary needle comprising a visualization marker (e.g., marker band) on a non-deployable region or portion of the needle. The exemplary needle in FIG. 20 may be incorporated into any of the examples herein, including in any incorporated by reference herein. For example, the needle in FIG. 20 may be one needle of a plurality of needles within a spine, and any of the needles within any number of spines of the apparatus may include a visualization marker (which may be attached to or unitarily formed therewith, for example).

FIGS. 21a and 21b are exemplary fluoro images showing needles of the exemplary apparatus in an undeployed state and a deployed state, respectively, wherein the apparatus comprises one or more needles that comprise visualization markers (labeled as “marker bands”). Each axially moveable rail assembly may comprise more than one needle, and less than all of the needles may include a visualization marker (e.g., only distal needles of any or all of the rail assemblies may comprise a visualization marker, as is labeled in FIGS. 21a and 21b).

FIG. 22 is an exemplary embodiment that includes a customer coupler to custom syringe arrangement.

FIGS. 23a and 23b illustrate exemplary optional rapid exchange configurations.

FIG. 24A is a perspective view of a distal portion of an exemplary agent delivery device, with an expandable member in an expanded configuration.

FIG. 24B is a perspective view of a distal portion of an exemplary agent delivery device that includes a multi-lumen, and which also illustrates an exemplary guidewire lumen.

FIGS. 25 and 26 are exemplary sectional views of exemplary multi-lumens, which may optionally be incorporated into any of the agent delivery devices herein.

FIG. 27 illustrates an exemplary apparatus in an expanded configuration, the apparatus including one or more primary spines with needles deployed, and one or more secondary spines.

FIG. 28 illustrates an exemplary secondary or primary spine, with a discontinuity in the wall of the spine, wherein the discontinuity may facilitate delivery of a second agent out of the spine.

FIG. 29A illustrates an end view of exemplary device in a deployed configuration with needles deployed, wherein the device includes a plurality of primary spines and a plurality of secondary spines.

FIGS. 29B and 29B′ illustrate an exemplary device with one or more primary spines and one or more secondary spines, wherein secondary spines are positioned in close proximity to the primary spines, adjacent to the primary spines (optionally, but not necessarily, contacting the primary spines).

FIG. 29C illustrates an alternative to FIGS. 29B and 29B′, in which first and second secondary spines are in close proximity to a single primary spine, wherein the first and second secondary spines are on different sides of the primary spine adjacent to the primary spine (optionally, but not necessarily, contacting the primary spines).

### DETAILED DESCRIPTION

The disclosure herein is related to methods, devices and systems for the delivery of diagnostic and/or therapeutic agents for the purpose of denervating nerves surrounding renal arteries. The devices and systems herein may be referred to as infusion devices and systems.

Infusion devices herein may include a plurality of deployable needles, which are spaced axially (also referred to herein as longitudinally) and circumferentially apart around the infusion device, allowing more uniform circumferential coverage and a greater span of tissue along the length to be targeted with a fluid agent without having to move the infusion device within the vessel. It is of course understood that any of the treatments herein may include delivering a fluid agent, after which the infusion device may be moved to a different location within the vessel or to a different vessel before again delivering the same or a different fluid agent.

Additionally, infusion devices herein may be positioned against a vessel wall upon application of a radially outward force, which is generally described herein as a force applied by an inflatable member or balloon, although it is conceivable that non-inflatable members may alternatively be used. After the infusion device is apposed against the vessel wall, the needles can be deployed outward such that they pierce through the vessel wall and into tissue surrounding the vessel. The desired therapeutic agent is delivered though the deployed needles, out of the needles, and into the target tissue to ablate nerves within the renal artery wall and/or surrounding the renal artery wall. In some methods, the volume and/or rate of infusion may be controlled based on one or more of a treatment zone, vessel inner diameter, and/or desired volume of agent infusion.

The ablative therapeutic fluids herein may be one or more agents adapted for chemical neurolysis, such as, and for example only, alcohol.

The disclosure that follows describes non-limiting exemplary ablation devices (any of which may be referred to herein as infusion devices) that are adapted and configured to deliver one or more ablative therapeutic agents and provide one or more of the advantages set forth herein, such as efficiently delivering a desired volume or dose to a target region of tissue within and/or surrounding the vessel wall, and in some cases from 1 mm to 8 mm away from the renal artery lumen, depending on the axial location at which the ablative agent is being delivered.

In this disclosure, the phrases “spine” and “infusion spine” (or similar phrases) refer to infusion spines that include at least one radial opening from which a needle is deployed.

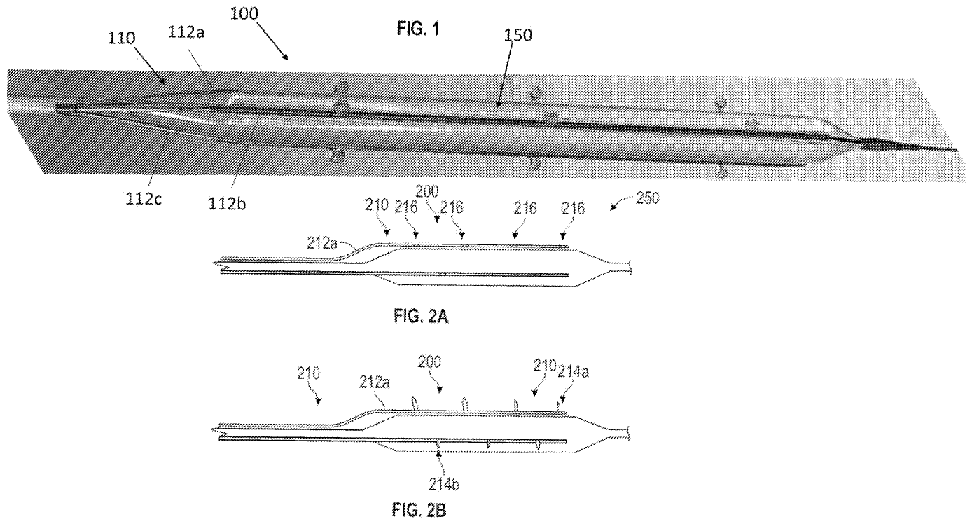

FIG. 1 illustrates a distal region of an example of an infusion device for renal denervation and incorporates by reference the entire disclosure of FIG. 1 from U.S. Pat. No. 11,071,847. Infusion device 100 includes an expandable infusion scaffold 110 that includes at least first and second infusion spines 112a, 112b, and 112c (three shown in this example), which are shown in FIG. 1 in expanded configurations with the infusion needles deployed. Unless indicated herein to the contrary, the infusion spines herein may also be referred to as a plurality of infusion spines, or plurality of spines. Infusion spines are sized, positioned, and configured to be expandable by a generally radially outward force, which in this example is applied by an inflatable member 150. Any of the inflatable members herein may include one or more of a compliant material (e.g., polyurethane or silicone), a non-compliant material (e.g., polyester or nylon), or a semi-compliant material. As shown, the infusion spines 112a, 112b and 112c are circumferentially spaced about an outer surface of the inflatable member 150 with a long axis (LA) of the infusion device when the spines are expanded. The long axis in this embodiment is also a long axis of the inflatable member 150. In this example, the spines are parallel (or substantially parallel) with the long axis of the infusion device 100 and the inflatable member 150 when expanded, as shown. As used herein, the phrase substantially parallel in this context includes slight deviations from being parallel and includes spines that have configurations that still facilitate the efficient and effective delivery of therapeutic agent to the desired tissue. One of skill in the art will appreciate that substantially parallel as used in this context allows for some deviation from strictly parallel, such as at an angle of five or ten degrees relative to a long axis, for example.

In this example the inflatable member has a cylindrical configuration when expanded, as shown. The term cylindrical as used in this context includes configurations that approximate a cylinder even if not perfectly cylindrical, which may be the case if a plurality of infusion spines are attached or engaging an outer surface of the inflatable member and the balloon does not have a perfectly cylindrical configuration when expanded. Additionally, an inflatable member may still be considered to have a cylindrical configuration even if the inflatable member has at least one end region that is tapered or has any other configuration that is not orthogonal with the long axis, such as the tapered distal and proximal ends of the inflatable member that are shown in FIG. 1. Additionally, the balloon may optionally have an expanded configuration that is slightly tapered in the proximal to distal direction. Additionally, for example, an inflatable member with a general dumbbell configuration may be considered to have a cylindrical configuration. Additionally still, when the description herein describes inflatable members having cylindrical configurations when expanded, it refers to the configuration the inflatable member would take after being expanded outside of a patient. This is meant to clarify that when expanded or inflated within a vessel of the patient, there may be one or more anatomical restrictions that prevent the inflatable member from transitioning to the cylindrical configuration it would assume if expanded outside of a patient, such as the configuration of the vessel wall in which the infusion device is placed. In both scenarios, the inflatable member in these examples is considered to have a cylindrical configuration when expanded.

The infusion spines herein may be connected (directly or indirectly) to the inflatable member, such as by bonding, adhesion, or using any other suitable technique for securing the spines to an inflatable member. In any of the examples herein, the spines may alternatively not be connected to the inflatable member, but they are still adapted to be expanded by inflation of the inflation member due to their proximity to the inflatable member. For example, the expandable infusion scaffold may be delivered on or over a balloon-based catheter in a compressed low-profile delivery state, and then expanded by dilating the balloon-based catheter at the intended location within the vessel.

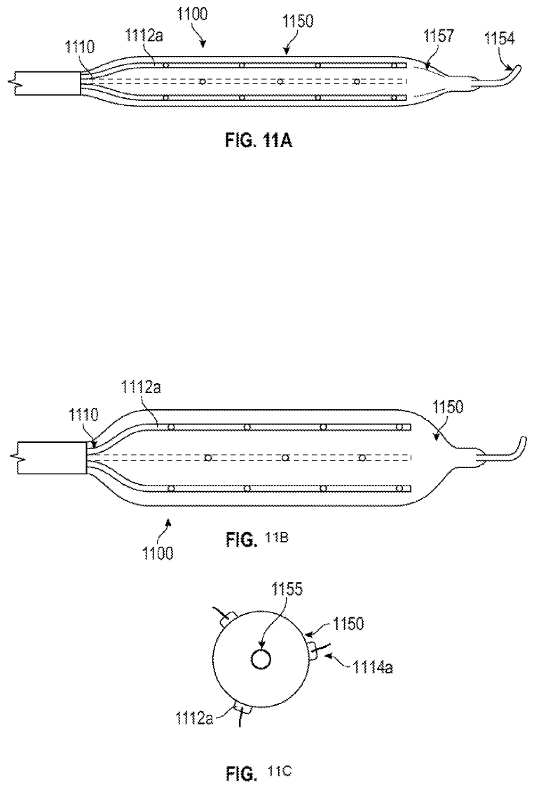

FIG. 1 shows an exemplary inflatable member 150 and an expandable infusion scaffold 110, both in an expanded state or configuration. For delivery, the expandable infusion scaffold is in a collapsed delivery configuration in which the infusion spines are closer to circumferentially adjacent spines than in the expanded state, such as shown in FIG. 11A. It is understood that FIG. 11A is meant to illustrate an infusion scaffold in a configuration in which it is not fully expanded (e.g., delivery configuration, or partially expanded). During delivery, the inflatable member is also in a lower profile unexpanded (and uninflated) collapsed delivery configuration. The internal volume of the inflatable member is also less in the delivery state than in the deployed state. Once the infusion device is delivered to the target location within a vessel, the inflatable member is inflated, which pressurizes the inflatable member. This expansion of the inflatable member causes the inflatable member to increase in a radial dimension and apply a force to the plurality of infusion spines that are disposed around the inflatable member. This causes the infusion spines to expand radially and which also causes the relative circumferential distance between the infusion spines to increase, an example of which is shown in FIG. 11B. The expandable infusion scaffold is thus expanded towards the vessel wall by inflating and expanding the inflatable member.

The inflatable member may have a variety of collapsed states or configurations. For example, the inflatable member may be folded in one or more locations to facilitate its collapse, while in other embodiments the inflatable member may not have a particular or well-defined collapsed state.

The inflatable members herein are sized and configured such that when expanded, the plurality of infusion spines will be moved radially outward and in contact or substantial contact with the vessel wall. It is understood that due to some variability in vessel wall size, some portion of any of the infusion spines may not make direct contact with vessel wall. The inflatable member may be sized such that it may have a deployed diameter that is larger than an intended vessel size to help ensure that the infusion spines are in contact or substantial contact with the vessel wall. Maintaining sufficient pressure in the inflatable member such that the infusion spines are in substantial contact with the vessel wall can help support the needles as they are deployed and pierce through the vessel wall, which is described in more detail below.

Any of the expandable scaffolds herein may have infusion spines that are optionally equidistantly spaced apart along their lengths, an example of which is shown in FIG. 1. For example, two infusion spines may be spaced apart 180 degrees around the inflatable member when the scaffold and infusion spines are expanded. Alternatively, three infusion spines may be spaced apart 120 degrees around the inflatable member when the scaffold and infusion spines are expanded. Alternatively, four infusion spines may be spaced apart 90 degrees around the inflatable member when the infusion spines are expanded, and so forth. In the collapsed delivery state, the infusion spines of the scaffold can also have the same general relative relationship even though they are closer together and not spaced as far apart.

While equal spacing between spines may in some applications provide more complete delivery of an agent to target tissue around or in the vessel wall, in alternative examples the infusion spines may not all be equidistantly spaced apart around the inflatable member.

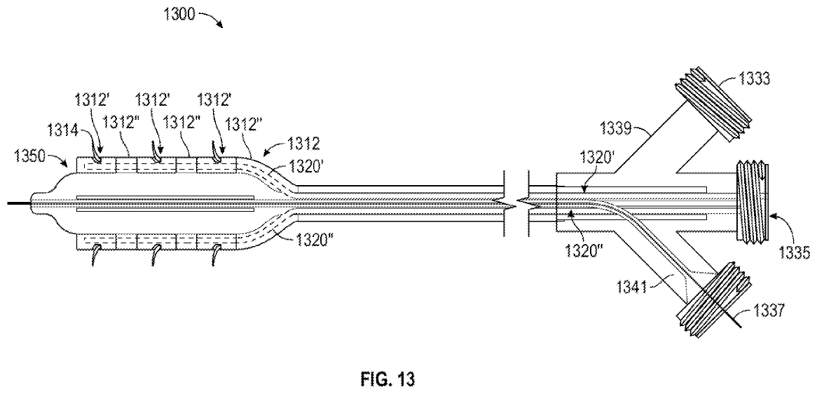

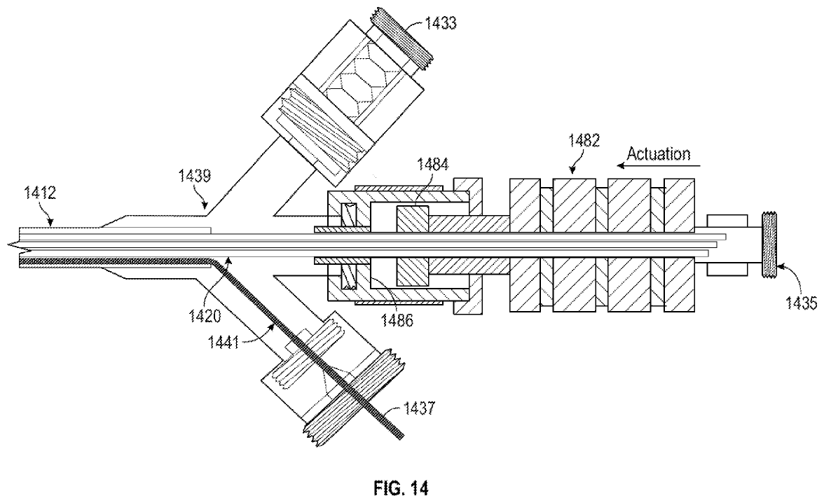

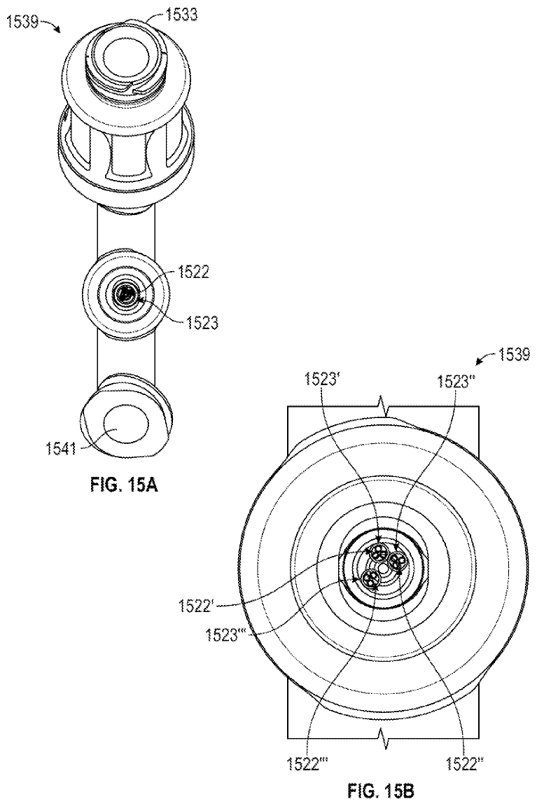

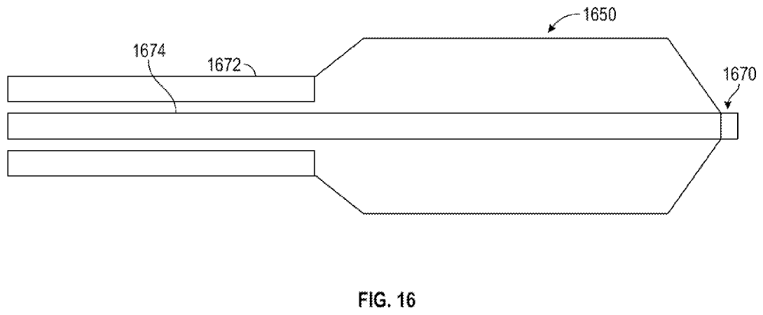

FIG. 16 illustrates a distal portion of an exemplary infusion device for renal denervation, wherein the expandable scaffold is not shown for clarity. In this example, the infusion device includes an inflatable member 1650, which is shown inflated. A distal end of inflatable member 1650 is coupled to inner shaft or member 1670, and a proximal end of inflatable member 1650 is coupled to outer shaft 1672. The inner and outer shafts 1670 and 1672 define therebetween inflation fluid pathway 1674, which is in fluid communication with an interior volume of inflatable member 1650. The inner volume of inflatable member 1650 and fluid pathway 1674 are in fluid communication with a fluid inflation port, such as inflation port 1333 or inflation port 1433 shown in FIGS. 13 and 14, and which are described in more detail below. Alternatively, the inflatable members herein may be secured to the infusion device in a manner that may be the same or similar to known balloon angioplasty catheters, examples of which are described in U.S. Pat. Nos. 4,782,834 and 10,086,175, and which are incorporated by reference herein for all purposes. Any of the fluid delivery devices herein that include one or more spines may include features shown in and described relative to FIG. 16.

Once the expandable inflation scaffold is expanded and in contact with (or at least substantially in contact with) or directly adjacent the vessel wall, each of a plurality of needles within a spine are deployed outward from a radial opening in the infusion spine, examples of which are shown in FIGS. 1 and 5. FIG. 1 illustrates a plurality of needles deployed from spines of the expandable infusion scaffold. In this merely illustrative example, there are three needles shown deployed from each of the infusion spines. In any of the embodiments herein, each infusion spine may be associated with from two to fifty needles, all of which can be deployed from a radial opening in the spine. As used in this context, the term “associated” refers to needles that are within any particular spine in a delivery state, and are deployable from that particular spine to pierce the vessel wall.

When this disclosure refers to an infusion spine, it is generally referring to one of the infusion spines of the expandable scaffold. Additionally, when a feature is described with respect to any particular or individual infusion spine, it is understood that all of the infusion spines of any particular scaffold may also have any or all of those features.

Sets of needles associated with any of the infusion spines herein are generally axially spaced apart, as shown in the examples of FIGS. 1, 2B and 5, for example. Spacing the needles axially apart can provide maximum coverage of the therapeutic agent along the length of the target tissue within and/or around the arterial wall (to target the nerves), which can increase the volume of tissue that may be targeted by using the infusion devices herein. Additionally, by having a plurality of infusion spines spaced around or about the device, with each infusion spine having a plurality of axially-spaced needles deployable therefrom, the infusion devices herein can ensure or increase the likelihood of delivering the ablative agent to as much target tissue within and/or around the vessel wall as possible without having to rotate or move the infusion device to provide the desired circumferential coverage of the infused agent. It is of course understood that the infusion devices herein may also be moved in between episodes of agent delivery into the vessel wall. In these instances, the needles may be retracted, and the infusion device can be moved to a different location within the vessel or to a different vessel. The inflatable member and the scaffold are generally collapsed (at least partially) before moving the infusion device to a new location. It is also understood that devices herein may include a single spine, for example in a helical configuration, wherein a plurality of needles are associated with the single spine.

In any the infusion devices herein, any two axially spaced needles associated with an infusion spine may be spaced from 1 mm to 40 mm apart, such as from 5 mm to 35 mm apart, such as from 10 mm to 30 mm apart, such as from 15 mm to 20 mm apart.

In some illustrative embodiments, any of the infusion devices herein may include from two to 50 needles total. For example, an infusion device with three spines, each associated with two needles, would have six needles total. Additionally, for example, an infusion device may include only one spine in a helical configuration that is associated with two needles (the proximal needle longer than the distal needle). Additionally still, for example, an infusion device may include only two spines, each associated with only two needles (the proximal needles longer than the distal needles), in which case the infusion device would include four needles total.

FIG. 1 illustrates an example in which infusion spines have the same lengths and have distal ends that extend as far distally as the other spine distal ends.

Needles in different spines may be axially aligned. For example, the exemplary needle placement in FIG. 1 shows three sets of needles that are axially aligned in three needle rows. A row as used in this context refers to two or more needles in different spines that are axially aligned.

In any of the infusion devices herein, the number of needles associated with each of the infusion spines may be the same. FIG. 1 shows an example of this, with three needles per spine. In alternatives, the number of needles in each of the infusion spines may not be the same. For example, one spine may be associated with two needles, while a second spine may be associated with three needles. Any of the infusion devices herein may have an expandable scaffold with a plurality of spines, optionally wherein none of the spines has the same number of needles as any other spine.

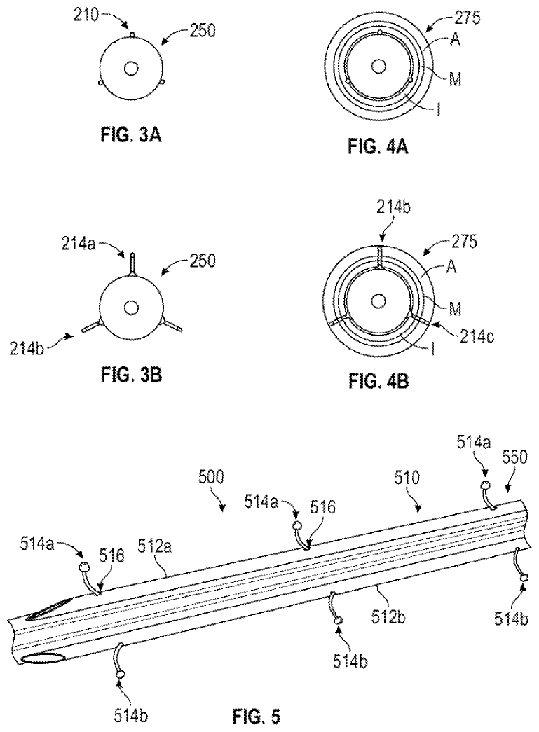

FIGS. 2A, 2B, 3A, 3B, 4A and 4B illustrate an exemplary infusion device 200 with an expandable infusion scaffold 210 that includes a plurality of infusion spines 212 (one labeled as 212a). Any suitable feature from FIG. 1 or described elsewhere herein may be incorporated into infusion device 200. Infusion device 200 also includes inflatable member 250 that when inflated and expanded causes the expandable infusion scaffold 210 to expand, described in more detail elsewhere herein. Each of the plurality of infusion spines includes a plurality of radial openings or windows 216 (shown in FIG. 2A), through which the plurality of needles 214 (labeled as 214a, 214b and 214c for the different spines) extend when deployed. FIG. 2A (side view), 3A (end view) and 4A (end view in an exemplary vessel 275) show the infusion device after the inflatable member 250 has been inflated but with the needles not yet deployed, while FIGS. 2B, 3B and 4B show exemplary needles 214 deployed through the openings in the infusion spines 212. FIG. 4B illustrates the needles 214 piercing into (which may be referred to as “through”) the vessel wall 275 and extending into the adventitia “A.” FIGS. 4A and 4B illustrate intimal “I,” medial “M,” and adventitial “A” layers of the vessel. Any other disclosure herein from any other example may be incorporated into the examples in FIGS. 2A-4B. It is understood that FIG. 4B illustrates a use to target vessel wall tissue, while other embodiments herein are targeting tissue within and/or outside of the vessel wall to target, for example, renal nerves that may reside within and/or outside of the adventitial layer of the vessel wall.

Generally, the infusion spines herein include a lumen and a plurality of openings or windows therein, such as openings 216 in FIG. 2A. The needles herein are generally disposed within an infusion spine in a delivery state in which the needle tips are radially constrained by the spine, and are deployed from the infusion spine out of one of the needle openings in response to axial movement (proximal or distal) relative to the spine to pierce the vessel wall. The needles herein may be disposed within and deployed from the infusion spines in a variety of ways. Additionally, the needles herein may be in fluid communication with a fluid source in a variety of ways. The needles herein associated with an infusion spine may be deployable at the same time. The needles herein associated with an infusion spine may be deployable by moving them together as a unit, such as if they are coupled to a common axially movable member within the spine. The needles herein associated with an infusion spine may be separately deployable from within the spine.

Each of the plurality of needles associated with an infusion spine may be coupled to an axially moveable member that is disposed within the infusion spine, such that axial movement of the axially moveable member relative to the infusion spine causes the axial movement of the needle relative to the infusion spine. For example, the needles may each be coupled to separate axially movable members such that the needles within a spine may be axially moved independently from one another.

In some embodiments herein, the needles associated with an infusion spine are all adapted to move together in unison upon the axial movement of an axially movable member, which may be referred to in this context as a common axially moveable member.

In some embodiments the axially moveable member (which may be referred to as a rail or rail track) is a separate structure that does not specifically define a fluid lumen, although in these examples the axially moveable member may house therein one of more fluid lumens that are in fluid communication with one or more needles. Additionally, in these embodiments, one or more fluid lumens within the axially movable member may also be moved axially relative to the infusion spine in response to axial movement of the axially moveable member.

FIG. 5 illustrates a portion of an exemplary infusion device 500, which may incorporate any of the disclosure related to infusion device 100 shown in FIG. 1 or any other feature described herein. Infusion device 500 includes an expandable infusion scaffold 510, which includes a plurality of infusion spines 512a, 512b (a third infusion spine 512c is not visible in the side view of FIG. 5). The infusion spines 512a and 512b each include a plurality of openings 516 through which the needles are deployed. In this example, each of the spines is associated with three needles as shown, but more or fewer may be associated with each infusion spine as is described elsewhere herein.

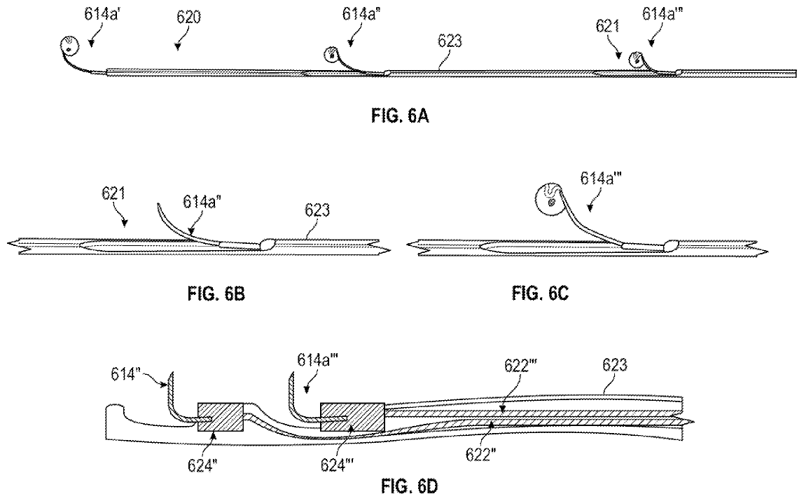

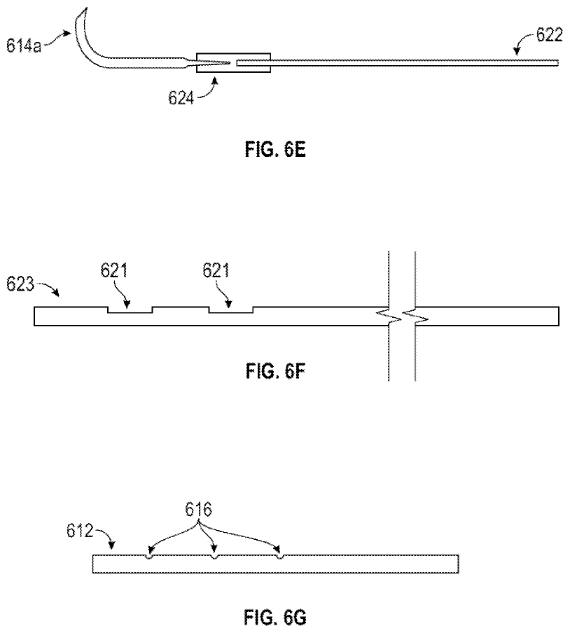

FIGS. 6A-6F illustrate exemplary features of an exemplary needle subassembly 620 (any of which may be referred to herein as a rail track subassembly, and vice versa), with the infusion spine not shown for clarity. Rail track subassembly 620 is configured to both move the needles to deploy them from the infusion spine openings, as well as provide housing for one or more fluid lumens that are in fluid communication with one or more needles, and such fluid communication to the needles to deliver the agent into the vessel wall when the needles are deployed from the openings in the infusion spine. FIG. 6E illustrates an exemplary needle 614a coupled to fluid lumen 622 with an optional coupler 624. In other embodiments any of the needles herein may be directly connected to a fluid lumen. The needle 614a and fluid lumen 622, as shown in FIG. 6E, are then positioned within rail 623, which is shown alone in FIG. 6F. Rail 623 is an example of an axially movable member that is configured to be axially moved to cause the axial movement of a plurality of needles. Rail 623 is also sized and configured to house therein one or more fluid lumens, in this case fluid lumen 622″ and fluid lumen 622′″, as shown in FIG. 6D. As shown in FIG. 6D, in this example each needle is in fluid communication with a distinct or individual fluid lumen, but they are coupled to rail 623 such that they move axially together in unison when rail 623 is moved. With respect to FIG. 6E, each needle is coupled to an individual fluid lumen as shown, then advanced through rail 623 and coupled thereto, as is shown in FIGS. 6A-6D . FIG. 6D illustrates one example of a plurality of individual fluid lumens 622″ and 622′″ housed or disposed within a lumen of rail 623. Rail 623, at least in this exemplary embodiment, can be moved axially to axially move all of the needles, as well as serve to house the individual fluid lumens therein.

The needle subassembly 623 shown in FIG. 6A can be then positioned in one of the infusion spines, such as by front loading or back loading. When the needle subassembly 620 is loaded into a infusion spine, the needles will deflect radially inward towards the openings 621 that are labeled in FIG. 6F, and the needle subassembly may be positioned in the infusion spine such that the needles and needle tips are just proximal to the infusion spine openings 616 (with the needle tips radially constrained by an inner surface of the spine), labeled in the exemplary spine 612 shown in FIG. 6G.

Any of the needles herein may be formed with a natural bias towards a deployed configuration in which the needles extend at least partially radially outward, such as is shown in FIGS. 6A, 6B, 6C, 6D and 6E. When the needles are collapsed radially down or inward for delivery, they may or may not have a perfectly linear configuration due to their naturally biased and curved deployed configuration. When collapsed for delivery, any of the needles may retain a slight curvature in their configuration, with their tips radially constrained by the inner surface of the spine.

The use of the term rail herein does not necessarily impart any structural limitations. The rails herein may be elongate members that are sized and adapted to be moveable within an infusion lumen to facilitate the movement of one or more needles. Any of the rails herein may be a tubular member or partial tubular member, such as rail 623 shown in FIGS. 6A-6F, or any other elongate member (with or without a lumen) that is sized and configured for axial movement within a spine.

As part of an exemplary manufacturing of a rail track assembly, the needle and corresponding fluid lumen may be front-loaded through the rail. A coupler (e.g., 624″ or 624′″), if used, may be secured (e.g., bonded, welded, or otherwise secured thereto) to the needle and fluid lumen as shown in FIG. 6E. The rail openings 621 may be formed by removing sections of the material of rail 623, which may itself be an elongate tubular member, such as a stainless steel or nitinol tubular member.

Each infusion spine in the exemplary infusion device shown in FIGS. 6A-6F is associated with at least three subcomponents or subassemblies-the infusion needle(s), the infusion lumen(s), and the rail track subassembly housing the respective infusion needle(s) and infusion lumen(s).

Any of the disclosure related to FIGS. 6A-6G may be incorporated by reference into any of the suitable disclosure herein related to devices and methods of use that includes scaffolds that comprise spines and radial openings.

In any of the examples herein, any of the fluid delivery lumens may have an outer diameter from 0.001 inches to 0.01 inches, for example. Fluid delivery lumens herein may also be referred to herein as fluid lumens.

In any of the examples herein, any of the axially moveable members (such as any of the rails) may have an outer diameter from 0.005 inches to 0.05 inches.

In any of the examples herein, any of the axially moveable members may have openings (e.g., openings 621) that are axially spaced from 5 mm to 80 mm apart, such as from 10 mm to 50 mm.

In any of the examples herein, any of the axially moveable members may have openings (e.g., openings 621) that have a length from 2 mm to 20 mm.

In any of the examples herein, any of the spines may have an outer diameter from 0.01 inches to 0.08 inches.

In any of the examples herein, any of the spines may have openings (e.g., openings 216, 516) that are axially spaced apart from 5 mm to 80 mm.

In any of the examples herein, any of the spines may have radial openings (e.g., openings 216, 516) may have openings with a diameter or length dimension from 0.05 mm to 10 mm.

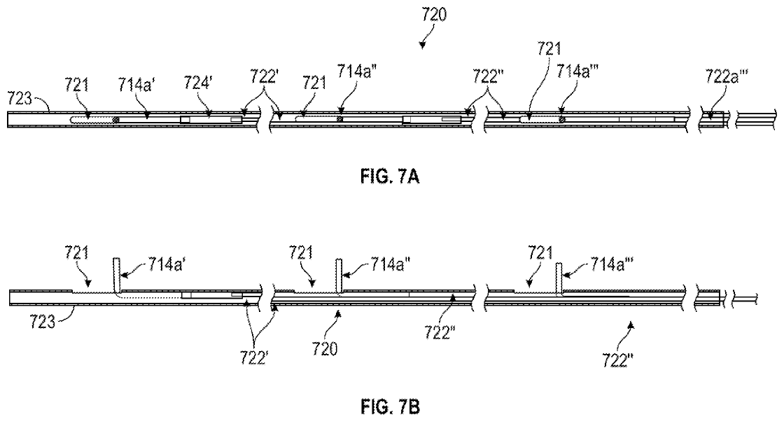

FIGS. 7A and 7B, in top and side views, respectively, illustrate an exemplary rail track subassembly 720 (spine not shown for clarity), with three exemplary needles in deployed configurations. Any of the features from assembly 620 of FIG. 6A may be incorporated into assembly 720. Rail track subassembly 720 includes rail 723, which has openings 721 therethrough (only one of which is labeled in FIG. 7A), and in this example there are three openings 721 in rail 723. Needles 714a are coupled to individual and distinct fluid lumens 722, optionally via couplers 724 but alternatively directed connected thereto, which may be secured to rail 723 to secure the needle to the rail 723 and provide unitary axial movement of the needles 714 (which are individually labeled as 714a′, 714a″, and 714a′″).

FIGS. 7A and 7B also illustrate how fluid lumens may extend through the rail 723 lumen. For example, fluid delivery lumen 722′ is in fluid communication with needle 714a′ and extends through rail 723. Fluid delivery lumen 722′ extends adjacent to central needle 714a″ and fluid delivery lumen 722″, as shown in the central regions of FIGS. 7A and 7B. In the proximal region shown in FIGS. 7A and 7B, all three fluid delivery lumens 722′, 722″ and 722′″ are adjacent to one another within the rail 723. Any of the fluid delivery lumens herein may include a bend or deviation in its path such that it can pass next to a different needle and its associated fluid delivery lumen, which is shown in FIGS. 7A and 7B. In this manner, the needles can extend in the same direction from the spine, which can be seen in the top view of FIG. 7A. In the top view of FIG. 7A, the needles are all extending upward, or out of the page.

Any of the disclosure related to FIGS. 7A and 7B may be incorporated by reference into any of the suitable disclosure herein related to devices and methods of use that includes scaffolds that comprise spines and radial openings.

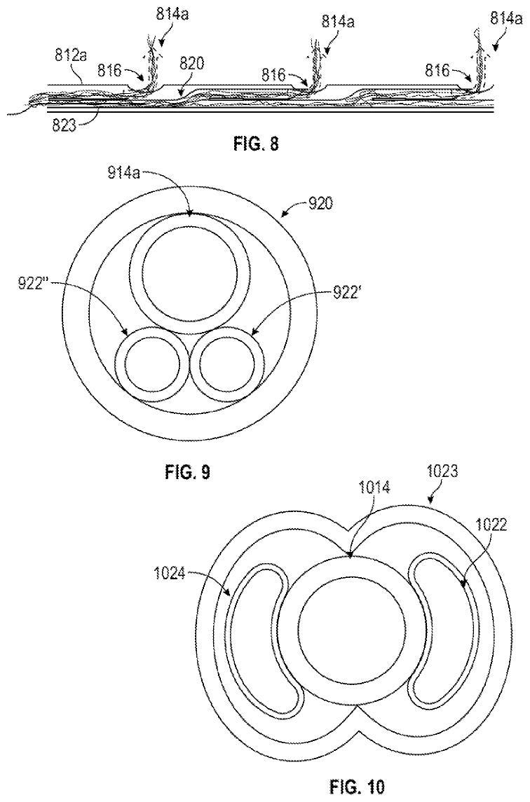

In some embodiments, the axially movable member may also at least partially define a fluid lumen that is in fluid communication with one or more needles, such as in the example shown in FIG. 8. FIG. 8 illustrates an exemplary needle assembly 820 shown within an exemplary spine 812a, which includes top or radially outward openings 816. Needle assembly 820 is an axially movable member that in this embodiment also at least partially defines a fluid delivery lumen as shown that is in fluid communication with all of the needles 814a. Needles 814a are shown in their deployed configuration (tissue not shown for clarity) extending out of the spine openings 816. Any other feature from any other example herein may be incorporated into the features shown in FIG. 8, including use with any other inflatable member herein. Any of the disclosure related to FIG. 8 may be incorporated by reference into any of the suitable disclosure herein related to devices and methods of use that includes scaffolds that comprise spines and radial openings.

FIGS. 11A-11C illustrate an exemplary embodiment of a device, wherein FIGS. 11B and 11C show side and end views, respectively, in a deployed configuration. An exemplary guidewire 1154 disposed within guidewire lumen 1155 is also shown, which may be used to deliver any of the infusion devices herein using known guidewire delivery techniques and methods.

Any of the disclosure related to FIGS. 11A-11C may be incorporated by reference into any of the suitable disclosure herein related to devices and methods of use that includes scaffolds that comprise spines and radial openings.

Any of the lumens herein (e.g., infusion spine lumen, rail lumen, and/or fluid lumen) may have or benefit from having one or more regions with sufficient flexibility to allow for the infusion device to be delivered to the target location in the vasculature. For example, any of the lumens herein may incorporate a tubular member having a wall with one or more regions with one or more discontinuities, such as cuts, therein (e.g., a laser cut or other technique) that imparts some degree of flexibility along at least a portion of its length. Discontinuities such as cuts made in a wall of any tubular member herein may be in the form of, for example without limitation, including combinations thereof, and at least partial spiral pattern, and at least partial brick pattern, or any other pattern that increases the flexibility of the wall of the lumen.

More than one pattern may be implemented in the wall of any lumen (spine lumen, rail lumen, fluid delivery lumen, etc.), and the shape or configuration of a cut pattern may change along the length of the lumen.

Any of the fluid lumens herein may optionally include a non-permeable membrane on one or both of an inside or the outside, such as an elastomeric membrane (e.g., urethane, silicone, or hydrogel), which can prevent fluid from leaking therethrough. For example, any lumens that may include or more discontinuities (e.g., cuts) therein (e.g., laser cut tubes) may include one or more membranes secured thereto to maintain integrity.

Any of the lumens herein may comprise, for example, any combination of nitinol, stainless steel, polymer tubing, polyimide, braided tubing, or other structural material. Any of the lumens herein may be constructed to provide the desired fluid integrity and/or flexibility when being delivered to the target delivery site.

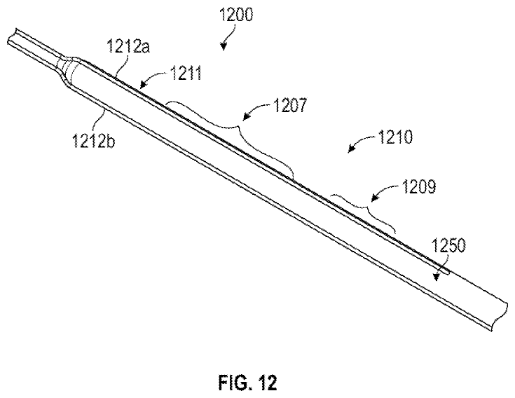

In some examples, sections of infusion spine(s) in between needle regions may be more flexible to provide more flexibility at those locations, while the spine regions where the needles are deployed may have relatively higher stiffness to aid the needle piercing through tissue or calcifications. FIG. 12 illustrates an exemplary infusion device 1200, with inflatable member 1250 and scaffold 1210 in expanded configurations or states. Scaffold 1210 includes a plurality of spines 1212a and 1212b. Infusion spine region 1207 may be configured to be more flexible than distal region 1209 and proximal region 1211 that are axially adjacent to region 1207. Needles may be present in regions 1209 and 1211, for example. Each spine may have a plurality of regions 1207 that are more flexible that other sections of the spine, any of which may be axially spaced apart with less flexible spine regions in between, which is described in more details with respect to FIG. 13. Any of the disclosure related to FIG. 12 may be incorporated by reference into any of the suitable disclosure herein related to devices and methods of use that includes scaffolds that comprise spines and radial openings.

FIG. 13 illustrates an exemplary infusion device 1300 shown with expandable member 1350 in an expanded configuration and a plurality of needles 1314 (only one of which is labeled) deployed from openings in spines 1312 (only one spine is labeled, and there may be additional spines and associated needles). In this example, the spines include first regions 1312′ at and around the locations where needles extend through openings therefrom, and regions 1312″ axially adjacent and optionally in between first regions 1312′. First regions 1312′ may be considered to include the spine openings from which the needles extend. First regions 1312′ may be less flexible than regions 1312″. This arrangement may provide sufficient stiffness to the spine region where the needle extends therefrom, helping the needle pierce through tissue (or calcifications), while regions 1312″ can provide more flexibility for tracking and delivery. Any of the spines herein may include first and second regions with different stiffness as in the example of FIG. 13.

As is set forth herein, the scaffold may or may not be attached to the inflatable member. In examples in which the scaffold (including the spines) is attached to the inflatable member, the spines may be secured to the inflatable member along their entire length, or less than their entire length. In some devices, the individual spines may be attached to the inflatable balloon at a plurality of axially spaced sections or regions along its length, and not directly attached to the inflatable member at one or more axially-spaced sections or regions along its length. For example only, with respect to FIG. 13, the plurality of spines may be attached to the inflatable member 1350 in regions 1312′, but not attached directly to the inflatable member 1350 in regions 1312″. Not directly attaching the spines to the inflatable member in regions 1312″ may allow for more movement and flexibility in the more flexible regions 1312″, which may provide more flexibility overall in the region of the scaffold, which can help when delivering the device. Any of the disclosure related to FIG. 13 may be incorporated by reference into any of the suitable disclosure herein related to devices and methods of use that include scaffolds that comprise spines and radial openings.

FIG. 13 also illustrates exemplary rail track or needle subassemblies 1320′ and 1320″ within corresponding spines, which may include a plurality of needles and one or more fluid lumens, which are described in more detail herein (there may be as many subassemblies as there are spines).

FIG. 13 also illustrates an exemplary proximal region of infusion device 1300. The proximal region includes an adaptor 1339, which in this example is a three-port adaptor. Adaptor 1339 includes an inflation port 1333 configured to couple to a fluid delivery device (e.g., Inflation Device commonly used with dilatation catheters) to deliver an inflation fluid to inflate expandable member 1350. Adaptor 1339 also houses a guidewire lumen 1341 therein, which is sized and configured to receive guidewire 1337 therein, which may facilitate delivery of any of the infusion devices herein over a guidewire. Adaptor 1339 also includes an actuator coupling region 1335, which may be sized and configured to couple to an actuation member, an example of which is described in more detail with respect to FIG. 14.

Any other feature from any other infusion devices herein may be incorporated into the example in FIG. 13, and vice versa.

FIG. 14 illustrates an exemplary proximal region of an infusion device, any features of which may be incorporated into any of the infusion devices herein. The proximal region includes optionally three-port adaptor 1439, which may house a guidewire lumen 1441 therein that is adapted to receive a guidewire 1437 therein for guidewire delivery. In this example, the proximal handle region includes an actuator 1482 that is in operational communication with the rail track subassemblies to facilitate axial movement thereof, which are generally labeled 1420, but it is understand there may be two or more (such as the three that are shown). The rail track sub-assemblies 1420 may have proximal ends that are attached (directly or indirectly) to an inner surface of actuator 1482, such as by using any suitable bonding technique, which thereby causes the rail track subassemblies to move distally upon distal actuation of the actuator 1482, to thereby deploy the needles from the spine openings. In this example, actuator 1482 has a plunger type construction, with a distal member 1484 that is sized to interface with inner surface 1486 to stop further movement of the actuator 1482. This stop mechanism is an example of a stop mechanism that is adapted to control the distal travel of the actuator 1482. This can be set at any desired distance to control the amount of needle deployment. The proximal portion also includes infusion port 1435, which is adapted to be coupled to a source of therapeutic agent to facilitate delivery thereof through the one or more delivery lumens and to the needles. A proximal region of an exemplary spine 1412 is also shown in FIG. 14, but it is understood that there may be as many spines as there are rail track sub-assemblies. Any other feature from any other infusion devices herein may be incorporated into the example in FIG. 14, and vice versa.

FIGS. 15A and 15B are proximal end views of the proximal region illustrated in FIG. 14, including three-port adaptor 1539, with FIG. 15B highlighting proximal ends of rails 1523 and fluid delivery lumens 1522 housed therein. FIG. 15A illustrates inflation port 1533 generally, guidewire lumen 1541 generally, and proximal ends of rails 1523 and fluid delivery lumens 1522 therein. FIG. 15B focuses on exemplary rails 1523′, 1523″, and 1523′″. In this example each rail 1523 houses therein three fluid delivery lumens, 1522′, 1522″, and 1522′″, respectively. The fluid delivery lumens are in fluid communication with the needles, such that a therapeutic agent may be delivered into the proximal ends of the fluid lumens 1522 and to the needles. Any other feature from any other infusion devices herein may be incorporated into the example in FIGS. 15A and 15B, and vice versa.

Any of the needles may be deployable using an external component (that remains outside the patient) that is operatively coupled to one or more needles of the infusion device. In some exemplary embodiments, all of the needles in the infusion device are deployable in unison, and may be operatively coupled to a common deployment actuator, an example of which is shown in FIG. 14 and described above. It is understood that other mechanisms may be used to deploy the needles, either in unison or not in unison. For example, the external portion (which may be referred to herein as a proximal region of the infusion device) may have more than one actuator, each of which may control a subsection of the plurality of needles.

Any of the needles herein may be referred to as microneedles, and may be comprised of nitinol, stainless steel, and/or a combination of nitinol, stainless steel, and other materials that adapt the needle to be able penetrate into the vessel wall. Any of the needles herein may range in length from 0.1 mm-6 mm (e.g., 1 mm-3 mm; 1 mm-2 mm; 2 mm-4 mm; 3 mm-4 mm) and in size from 20 gauge to 38 gauge, for example. For clarity, the lengths and/or size of individual needles may vary relative to any adjacent needles, either in the same spine or different spines. Furthermore, the relative inner diameter, outer diameter, and wall thickness of the individual needles may be uniform relative to adjacent needles, or they may vary relative to any adjacent needles, either in the same spine or different spines. Additionally, any of the needles herein may have at least one of an inner diameter (“ID”) and an outer diameter (“OD”) that varies along the length of the needle.

Any of the expandable infusion scaffolds herein may be configured to be an integral part of the balloon system. Alternatively, any of the expandable scaffolds herein may be configured as an independent structure that works ‘in synergy’ with a balloon-based system but is not attached to the balloon system and is not integral to such. As is described elsewhere herein, and incorporated into these embodiments, the expandable scaffold may take the form of various potential configurations designed to enable infusion lumen structural support and communication with the microneedles while also facilitating circumferential and longitudinal infusion of the intended agent to the target site.

In any of the infusion devices herein, the expandable infusion scaffold may comprise one or more infusion lumens extending in a longitudinal (axial direction; proximal-distal) or non-longitudinal pattern along at least a portion of the length of the balloon that is either integral to, or to be used in synergy with the infusion scaffold. Longitudinal in this context refers generally to at least a portion of an infusion lumen that is parallel with a longitudinal axis of inflatable balloon. In some embodiments, the scaffold may comprise one or more infusion lumens extending in a non-longitudinal pattern along at least a portion of the length of the balloon that is either integral to, or to be used in synergy with the infusion scaffold. Any of the infusion lumens herein may have one or more portions that extend longitudinally and one or more portions that extend non-longitudinally. Examples of a non-longitudinal configuration or pattern in this context include a spiral or helical configuration or other non-longitudinal pattern. For the sake of illustration, the following describes infusion lumens that run or extend longitudinally (axially) along at least a portion of the length of the scaffold. “Longitudinally” (and derivative thereof) and “axially” (and derivatives thereof) are generally used synonymously herein. “Linear” may also be used with longitudinal and axial when made in reference to a linear longitudinal or linear axial configuration, such as if parallel to a longitudinal (or long) axis of the infusion device or an inflatable member.

In some exemplary embodiments herein (such as in FIGS. 6A-6F), the microneedles are secured (e.g., directly attached, or attached via one or more intermediate components) to a rail or other elongate member that is loaded into and disposed in the infusion spine. Exemplary benefits of this design include, but are not limited to, 1) protection of the balloon, guide catheter, delivery sheath, vessel wall, or any other structure in proximity to the microneedles by isolating the sharp needle points during delivery to the target site and/or removal from the target site; 2) the ability to use the scaffold to facilitate controlled expansion of the infusion scaffold; and/or 3) added structural support during deployment of the needles. Needles that are secured to tracks or other elongate members herein may also enable the depth of needle deployment to be controlled or adjusted. For example, any of the rails herein may be in operable communication with an external portion (e.g., as shown in FIGS. 13-15B), wherein one or more actuators (e.g., rotatable knobs, axially movable sliders) in the external portion may be adapted to be actuated to control the relative degree of motion of the rail track subassembly (e.g., axial translation), and thereby control the length of the needles that exit radially or somewhat radially outward from the infusion spine.

Any of the rails herein may also be referred to as a support shaft, any of which may be solid or have a lumen therein. The rails herein may be made of any number of potential materials such as nitinol or stainless steel onto which the needles can be bonded or attached (directly or indirectly), and which may optionally be slatted or laser cut along at least a portion thereof to provide enhanced trackability. Additionally, any of the rails herein may be comprised of more than one type of material along the length of the device. Any of the individual needles herein may include a first end that may be straight or linear and the other free end may be pre-formed (e.g., heat set) to take a perpendicular or near perpendicular configuration (e.g. 60-120 degrees) to the surface of the vessel when the needle is in its deployed state. A straight or linear section of a needle may be individually secured (e.g., directly attached) to an axially moveable member such as a rail, allowing the free end to be free to deform and assume its deployed shape (e.g., pre-set shape) as it exits the infusion spine opening.

Axial spacing between needles may be optimized based on the desired anatomical coverage of the agent within and/or outside of the vessel wall, along with spacing to facilitate optimal delivery and trackability of the infusion device to the target site.

As described elsewhere herein, the individual rail remains inside the respective infusion spine, serving as a mechanism by which to advance and retract the microneedles. One or more openings (or windows) in the infusion spine provide guidance (or a pathway) for the microneedle(s) to exit the infusion spine and can also be adapted to function as added structural support as the needle penetrates into the vessel wall. Any of the infusion spines and/or rails herein may comprise or more structures or features configured to function as an additional intraluminal guide, alignment feature(s), and/or ramp as the needle advances out of the infusion spine opening, examples of which are described in WO2022/232589, published Nov. 3, 2022, any of which may be incorporated into any of the devices herein.

In any of the examples herein, advancement and retracting of one or more rails or support shafts, to which one or more microneedles are secured (directly or indirectly), may be enabled through a mechanical turn dial (or any other rotatable handle actuator) or any other mechanical actuation mechanism with intuitive settings to guide the user during deployment and retraction of the microneedles.

In any of the examples herein, after the microneedles are deployed, infusion may be initiated using, for example only, a controlled mechanism of volume delivery based on the target length and desired volume of agent infused.

In any of the examples herein, the number of needles per infusion spine may be of any desired number, inclusive but not limited to the range of two to fifty microneedles per infusion spine. In some embodiments, the microneedles may be attached or otherwise secured by techniques such as welding, soldering, mechanical crimping, adhesive, or other techniques to a rail and/or fluid delivery lumen. The needles herein may be bonded directly to a fluid delivery lumen, or they be bonded to one or more intermediate elements such as a coupler. Further, as is described in more details elsewhere herein, the depth of needle deployment may be controlled or adjusted, for example, by utilizing one or more controls in an external portion of the device that may be adapted to control the relative degree of motion of the rail track or support shaft subassembly and thereby control the length of needle that exits radially or somewhat radially outward from the device.

In some examples herein, each needle associated with a spine is in fluid communication with an individual and separate fluid delivery lumen along at least a portion of the catheter length.

Any of the fluid delivery lumens herein may have one of a variety of cross-sectional shapes inclusive of, but not limited to, round and kidney shaped. This may be done to help reduce the overall profile of the needle assembly without compromising the volume of agent that can be infused through the lumen(s). FIGS. 9 and 10 are exemplary and non-limiting sectional views of fluid delivery lumens.

Any of the lumens herein may be comprised of one or more materials inclusive of, but not limited to, polyimide, polymer, nitinol, composite, and/or combination thereof. Any of the fluid delivery lumens and needles within a rail may be secured using a variety of potential techniques such as, without limitation, crimping, welding, soldering, potting, adhesive, or other techniques inclusive of a combination thereof. In any of these embodiments, any single needles may thus be in fluid communication with a unique or distinct fluid delivery lumen that is only in fluid communication with that particular needle and not any other needles. In alternatives, a plurality of needles may be in fluid communication with a first fluid delivery lumen, and a different needle may be in fluid communication with a second fluid delivery lumen.

In any of the embodiments herein wherein the expandable scaffold is attached to the inflatable member, the scaffold and/or individual spines may be bonded to the balloon or secured to the balloon with one or more additional thin-walled layers of material, for example a polymer jacket and adhesive.

As disclosed elsewhere herein, in any of the embodiments herein, the infusion scaffold may be independent from the expansion balloon (not integrated therewith), yet is adapted to function in synergy with the expansion balloon. In these embodiments, the scaffold may be deployed prior to inflation of the balloon. For example, upon retraction of an outer scaffold sheath, the scaffold may be adapted to be self-expanding, partially self-expanding, or non-self-expanding. The expansion balloon may be then advanced within the scaffold and dilated to continue to or fully expand the infusion scaffold. The scaffold structure may be deployed passively by retracting an outer sheath (as would a self-expanding stent) or by a mechanical means activated in the handle of the device. The infusion scaffolds herein may be compatible with any off-the-shelf angioplasty balloon, and the balloon may optionally be drug-coated or uncoated. In some of these embodiments, the scaffold may be pre-loaded onto the expansion balloon (yet not attached thereto), with both delivered to the target site in unison, and the infusion scaffold may then be expanded as the dilatation balloon is expanded. The scaffolds herein may thus be at least partially deployed with an expansion balloon, but need not be bonded thereto.

In alternative examples, the scaffolds herein may be independent without the use of an expansion balloon. For example, the scaffold may be deployed into a target vessel and expanded radially. Radial expansion may be accomplished passively by retracting an outer sheath (as would a self-expanding stent that is commonly used in the field) and/or by a mechanical mechanism activated in the handle of the device. In an exemplary embodiment, the infusion scaffold is configured and adapted to be expanded using a mechanical mechanism or approach that compresses parts of the infusion scaffold longitudinally. The needles may then be advanced, as is described in more detail herein.

In some methods of use, the expandable scaffolds herein may be delivered about an inflatable member, either attached to the balloon or not. After the inflatable member and scaffold are delivered to the target location within a vessel, an inflation can be delivered to an inner volume within the inflatable balloon to cause its expansion. This balloon expansion applies a force to the expandable scaffold, causing the scaffold and spine to radially expand towards the vessel wall. The balloon can be expanded until the infusion device makes contact with the vessel wall. The needles may then be deployed from the spine opening and through the vessel wall, which is described in more detail elsewhere herein, and optionally by distally advancing one or more rails within the spines. The agent may then be delivered from a fluid source, through the one or more fluid delivery lumens, and out of the one or more needle ports and into the tissue within and/or surrounding the vessel wall optionally including the adventitia and surrounding tissue to ablate the renal nerves. The needles may be retracted by retracting one or more rails, and the scaffold and inflatable member may then be collapsed. The infusion device may then be recaptured (e.g., within a sheath or guide catheter) within a delivery sheath and removed from the patient or delivered to another location for a subsequent agent delivery process.

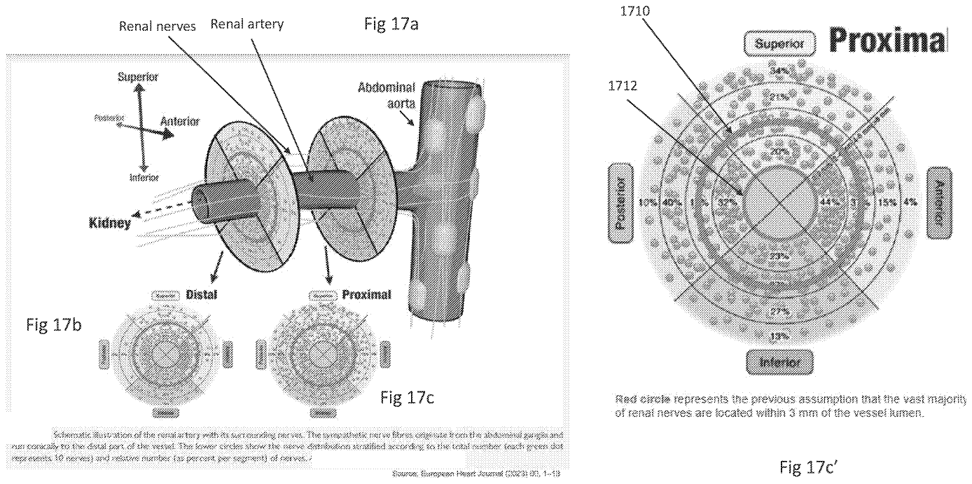

An additional aspect of this disclosure is related to methods of, and devices and systems adapted for, renal denervation. This aspect of the disclosure includes devices that are adapted to deliver an ablative fluid to nerves within and surrounding a renal artery to ablate the renal nerves and treat hypertension. FIG. 17a is a perspective view that represents an anatomical region that includes an abdominal artery, a renal artery branching therefrom towards a kidney (not shown), and renal nerves surrounding the renal artery. Renal denervation approaches have attempted to ablate the renal nerves surrounding the renal artery to treat hypertension using a device positioned within the renal artery. FIGS. 17b and 17c illustrate, at different axial locations as represented in FIG. 17a, the renal nerve distribution stratified according to the total number and relative number of renal nerves. FIG. 17c′ is an enlarged image of FIG. 17c, showing a thicker red circle 1710 which represents a previous assumption that the vast majority of renal nerves are located within 3 mm of the renal artery lumen (wherein the lumen is represented as the thinner red circle 1712). As can be seen in FIG. 17c′, there are many renal nerves beyond 3 mm from the vessel wall 1712. Distances zones can be more clearly seen in FIG. 17c′, including regions of 0-2 mm, 2-4 mm, 4-6 mm, and >6 mm from the lumen wall 1712. Additionally, comparing FIGS. 17b and 17c illustrates that at the axial location shown in FIG. 17c (which is a section at the proximal region in FIG. 17a), there are more nerves further from the lumen wall 712 than at the more distal location shown in FIG. 17b.

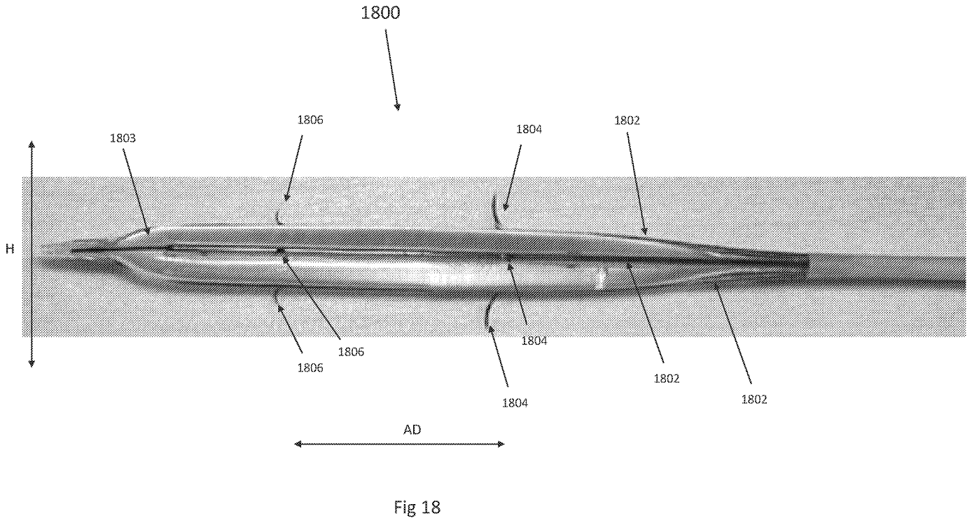

FIG. 18 illustrates an exemplary ablation device that optionally incorporates any of the features from any of the infusion devices herein, alone or any combination thereof. Based on the anatomical learnings shown in FIGS. 17a-17c′, ablation device 1800 may be particularly suited to more effectively ablate renal nerves. Device or apparatus 1800 includes an inflatable balloon 1803, shown expanded in FIG. 18, and spines 1802 disposed about the balloon 1803, as shown. Spines 1802 may include any of the features of any of the spines herein. One difference between device 1800 and alternative infusion devices herein is that device 1800 shows a first set of needles 1804 (three are shown) that, when deployed, extend further radially outward than a second set of needles 1806 when deployed (as shown in FIG. 18). The first set of needles 1804 is axially spaced (proximally in this case) from the second set of needles 1806. This causes device 1800, when in use, to deploy needles at different depths along the length of the renal artery with a single device placement. This facilitates delivering targeted denervation tailored to changes in radial distribution and concentration of nerves along the length of the renal artery, as shown in FIGS. 17a-17c′.

Proximal needles 1804 may extend radially from the radial opening of the respective spine, when in their fully deployed configurations, a distance from 1.5 mm-6 mm, as measured in the height dimension “H” shown in FIG. 18 (orthogonal to a long axis of the device), optionally from 2 mm to 4 mm.

Distal needles 1806 may extend radially from the radial opening of the respective spine, when in their fully deployed configurations, a distance from 0.5 mm-4 mm, optionally from 1 mm to 3 mm.

Proximal needles 1804 are an example of a set of needles that are axially aligned (in the side view shown in FIG. 18) in a proximal row when deployed, and distal needles 1806 are an example of a set of distal needles that are axially aligned (in the side view shown in FIG. 18) in a distal row when deployed. An axial distance or spacing “AD” between needle rows (labeled in FIG. 18) may be from 5 mm to 20 mm.

A distal needle 1806 and a proximal needle 1804 that are associated with one of the plurality of spines may be coupled to a rail that is axially movable within and relative to the associated spine (discussed above), such that the two needles are adapted to be axially moved together relative to and within the associated spine upon axial movement of the rail. The rail assemblies may be adapted such that the distal and proximal needles are adapted to be pushed distally together relative to the associated spine to the deployed configurations upon distal movement of the rail, or they may be adapted to be pulled proximally together relative to the associated spine to the deployed configurations upon proximal movement of the rail.

The plurality of spines may be arranged about the outer surface of the balloon in any arrangement described herein, such as a helical arrangement or an axial (longitudinal) arrangement.

Spines 102 may be secured to the inflatable balloon along at least a portion of the lengths, in any of the manners described herein, or they may not be secured to balloon.

A length of balloon 1803 where it is configured to be apposed against a renal artery wall may be from 10 mm to 40 mm, such as 20 mm to 30 mm, such as 25 mm.

Device 1800 may include no more than two rows of axially spaced needles, as shown, and optionally includes only six needles (three relatively shorter distal needles and three relatively longer proximal needles).

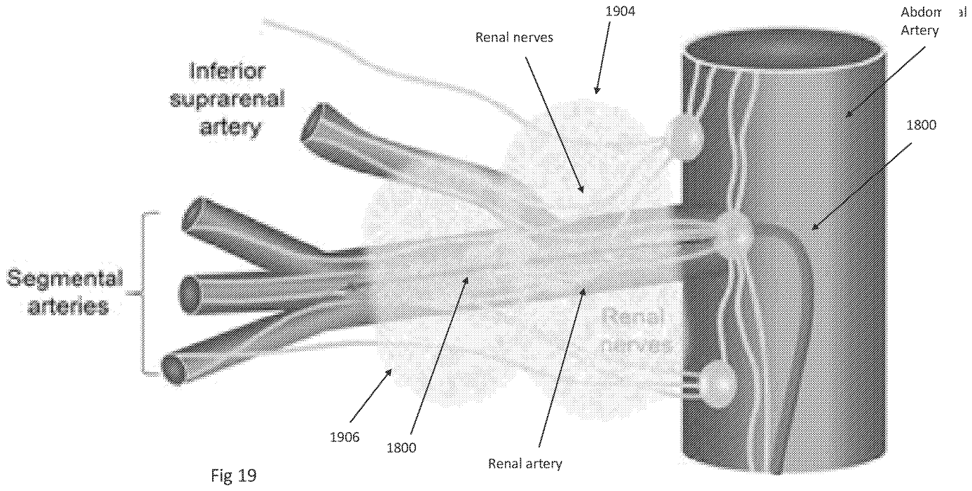

FIG. 19 represents part of an exemplary method of using device 1800, which is shown in FIG. 18. After balloon 1803 expansion, causing the spines 1802 to be moved toward the wall of the renal artery, and deploying the two sets of needles 1804 and 1806, FIG. 19 also represents first generally circumferential ablation zone 1904 after an ablative agent is delivered from the first set of (relatively longer) needles 1804, and second generally circumferential ablation zone 1906 after an ablative agent is delivered from the second set of (relatively shorter) needles 1806. As shown, the ablation zones 1906 and 1904 can overlap to some extent and are not necessarily distinct zones or regions. The ablation zones 1906 and 1904 can together create a total renal denervation ablation zone or region. The longer needles 1804 deliver an ablative agent into tissue where many nerves are located, as shown in FIG. 17c and FIG. 17c′ (e.g., up to 8 mm from the renal artery). The shorter distal needles 1806 deliver ablative agent at a different axial location to target shallower tissue where many nerves are located, as illustrated in FIG. 17b. Device 1800, by being adapted to deploy needles to different depths at different axial locations (optionally axially spaced from 5 mm to 15 mm apart) along the length of the renal artery, a single device placement facilitates targeted denervation specific to changes in radial distribution and concentration of nerves along the length of the renal artery. Device 1800, by targeting different tissue depths at different axial locations, device 1800 can more effectively ablate a greater number of renal nerves that extend along and outside of the renal arteries.

The methods of renal denervation herein may include using the devices herein to deliver one or more neurolytic agents to the renal nerves to thereby ablate the renal nerves. For example only, the methods of renal nerve ablation herein may include delivering ethanol, guanethidine, vincristine, or other neurolytic agent to the renal nerves using any of the devices or apparatuses herein. The entire disclosure of Seward, K. (2019). Adventitial Guanethidine (MMS-008) in Comparison to Ethanol and a Prior Formulation of Guanethidine (MMS-007) for Renal Denervation: Preclinical Norepinephrine and Histopathology Results. Journal of the American College of Cardiology, 74(13), Suppl B., is incorporated by reference herein in this regard. The entire disclosure of Stefanadis, C. (2013). Chemical Denervation of the Renal Artery with Vincristine for the Treatment of Resistant Arterial Hypertension: First-in-Man Application. Hellinic Journal of Cardiology, 54, 318-231 is also incorporated by reference herein in this regard.

This disclosure includes additional embodiments below, which are not necessarily limited to devices for use in renal nerve ablation. For example, one or more features in any of the examples below may have application with any of the devices and the methods of use described in WO2021/133966 A1, WO 2022/182598 A1, and WO 2022/232589 A1, which are fully incorporated by reference herein for all purposes.

One aspect of the disclosure is related to radiopaque needle marker bands (more generally described and used herein as needle visualization markers) may be integrated with and incorporated into any and all of the devices, systems and/or apparatuses herein (and any of their methods of use), including those incorporated by reference herein. It is thus understood that the visualization markers may be incorporated into devices and apparatuses regardless of the medical procedures for which they are used (e.g., on the veinous side, on the arterial side, for DVT, for PAD, for RDN, used in different bodily lumens, etc.).

To assist with visualization of needle movement and/or deployment, radiopaque marker bands may optionally be affixed to a portion of the infusion needles. As the rail track/needle assembly is moved axially (e.g., distally advanced), the radiopaque visualization marker provides visual confirmation of the rail-track/needle assembly advancement and/or positional information. Any of the needles in any apparatuses herein may comprise a visualization marker (or a plurality of visualization markers), which may be coupled to the needle during assembly, or which may be unitarily formed with the needle.

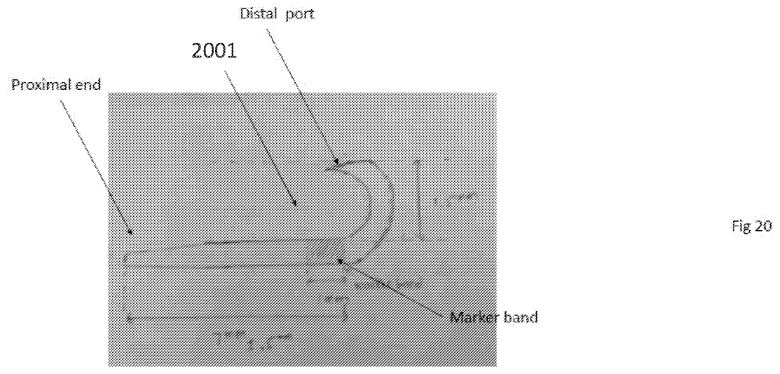

FIG. 20 represents a single needle 2001 in an exemplary deployed configuration, including a proximal end and a distal port or opening. Any of the needles herein may incorporate any feature shown in or described with respect to FIG. 20. As shown, the visualization marker (labeled as “marker band”) may optionally be coupled to (or unitarily formed with) a portion of the needle that is just proximal to the heat-set curve of the needle, and therefore does not exit the infusion spine opening nor penetrate the vessel wall (or other tissue) as the needle is advanced. Merely exemplary and non-limiting dimensions are shown in the side view of FIG. 20, including the exemplary length of the exemplary visualization marker (e.g., 1 mm). The exemplary radial dimension of the deployable portion of the needle is shown as 1.5 mm, as is the exemplary length (e.g., 7 mm +/−5 mm) of the needle portion that is proximal to the deployable portion (which includes the visualization marker).

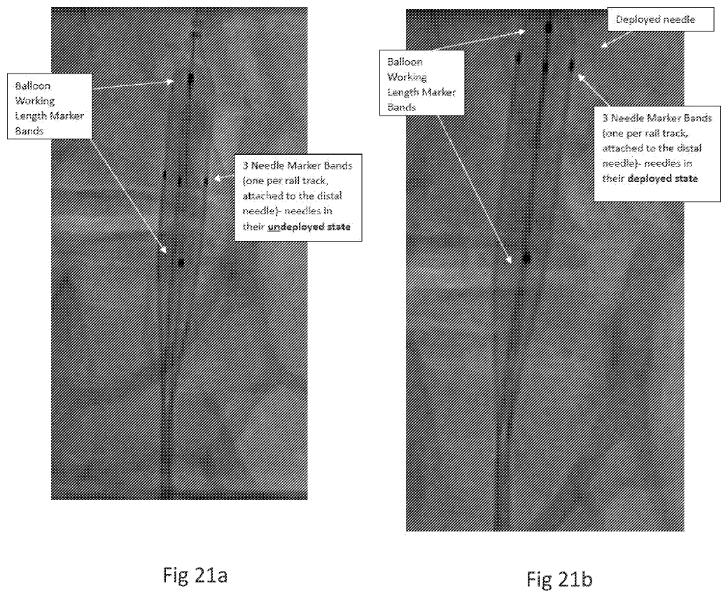

As the needles (and/or rails) are moved axially (e.g., advanced or retracted), the radiopaque marker bands on or incorporated into the needles can be seen under fluoroscopy, as shown in the fluoroscopy images in FIGS. 21a and 21b.

In any of the examples and embodiments herein, including in any of those described in WO 2022/232589 A1 (which is fully incorporated by reference herein for all purposes), the inflatable member (e.g., balloon) may include at least first and second different materials. For example only, the balloon may include an inner layer of a first material and an outer layer of a second material that is different than the first material, wherein the layers are directly or indirectly coupled. Without intending to be limiting, it may be beneficial to include an inner layer of a first material with certain properties and an outer layer of a second material with certain properties different than those of the first material. For example, and without limitation, in any of the embodiments herein, an inner layer may comprise or consist of nylon (which may be relatively more stable and/or stronger as an inflatable member) and an outer layer may comprise or consist of polyurethane (for example only), which may facilitate a relatively stronger adhesive or thermal bond to one or more sleeves (e.g., a polyurethane sleeve, a PEBAX® sleeve, other polymeric materials, etc.) that are around the individual spines and that are bonded to the inflatable member (in those embodiments herein that include one or more sleeve(s).

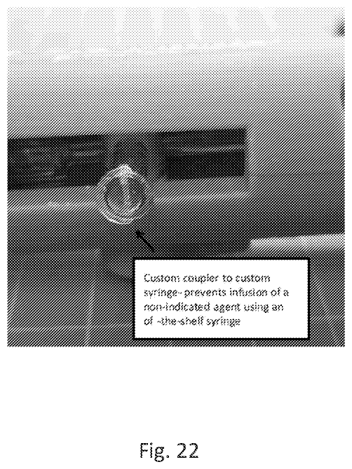

One aspect of the disclosure includes a handle that includes a custom coupler as shown in FIG. 22. The custom coupler as shown is a custom coupler that is adapted and sized to be coupled to a custom syringe, which prevents infusion of a non-indicated agent using an off the shelf syringe. Any of the proximal handles herein may include a custom coupler.

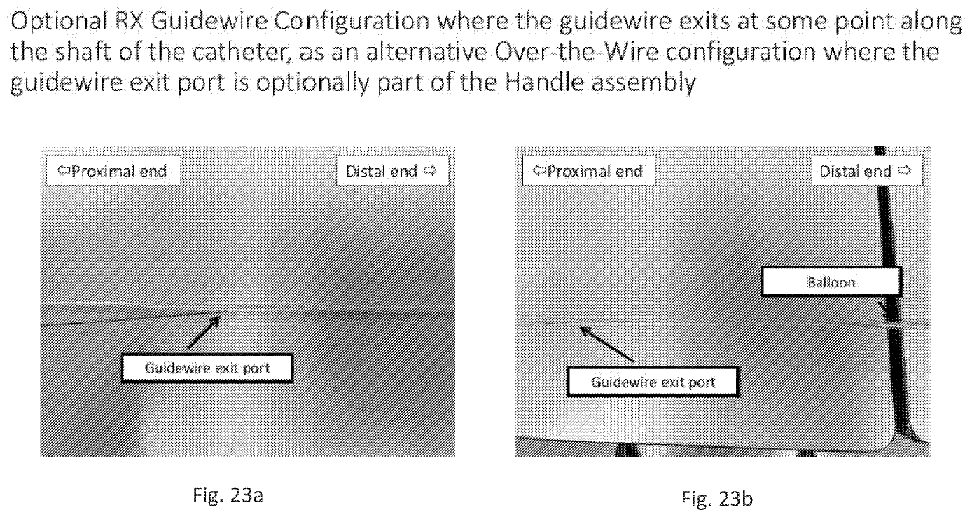

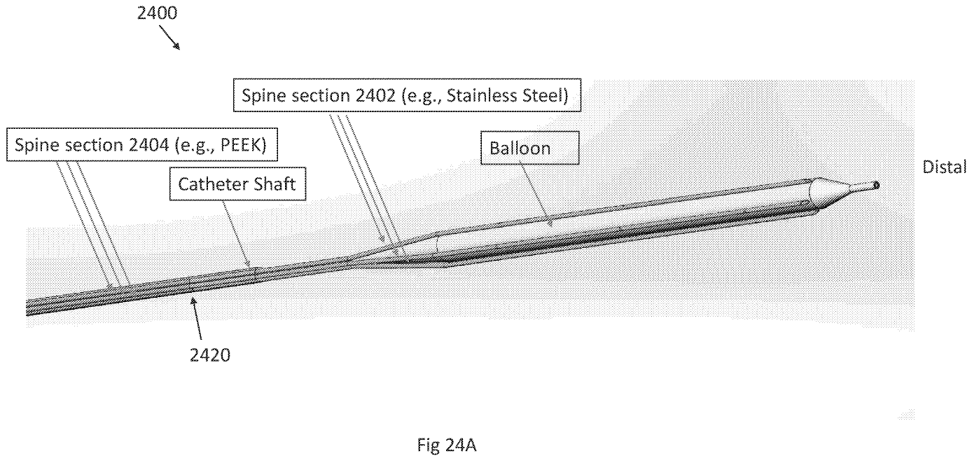

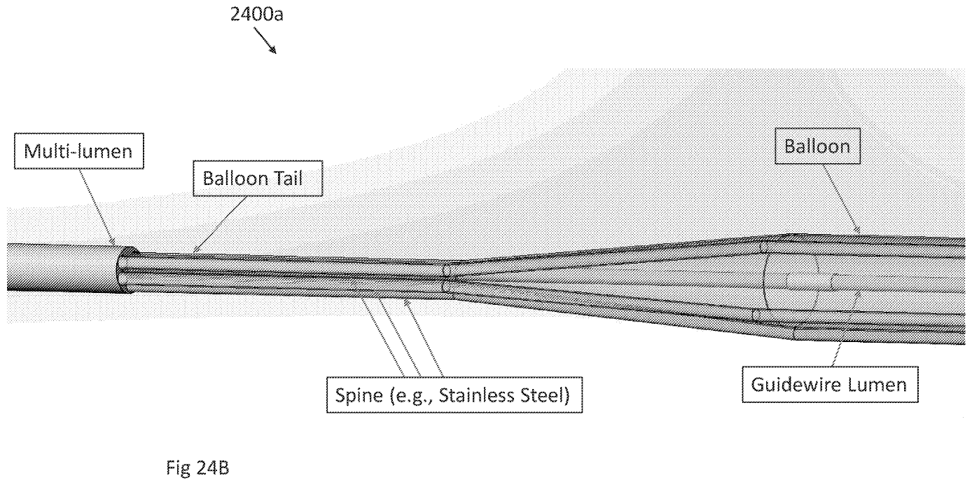

One aspect of the disclosure is related to optional rapid exchange guidewire configurations, exemplary details of which are shown in FIGS. 23a and 23b. The rapid exchange design may be integrated into any of the devices and systems herein.

FIG. 24A illustrates a distal region of an exemplary agent delivery device 2400, which may include any suitably combinable feature of any of the other devices and apparatuses herein (e.g., one or more spine securing members, needle assemblies, etc.), regardless of the described method of use. Device 2400 in FIG. 24A includes an inflatable balloon, shown in an expanded configuration, as well as an expandable scaffold that includes a plurality of spines, distal regions of which are shown expanded (optionally by balloon expansion, or expanded without needing balloon expansion). A plurality of radial openings in each of the spines are also shown (optionally in a curved outer surface thereof, as shown), optionally out of which needles or a secondary agent may be deployed. While not shown, device 2400 optionally includes a plurality of movable needle assemblies, each of the optional plurality of movable needle assemblies movable within and relative to the corresponding infusion spine, examples of which are described throughout this disclosure.

Any of the devices herein that are adapted to deliver one or more agents (even if they are named differently, such as “catheter” or “apparatus”) may include one or more spines with sections that comprise different materials, a mere example of which is illustrated in FIG. 24A. The spines of device 2400 each include at least first section 2402 and second section 2404, wherein first section 2402 is distal to second section 2404. Each first section 2402 comprises a first material and each second section 2404 comprises a second material. First and second sections 2402 and 2404 may be coupled directly or indirectly to each other, and optionally there may be one or more intermediate section (not shown) axially in between and separating the first and second sections. There may be advantages for first section 2402 to be made of a first material and second section 2404 to be a second material different than the first material. For example only, first sections 2402 may be an alloy such as stainless steel, with one or more laser cuts therein to impart flexibility (described elsewhere herein). There may be advantages for second sections 2404 to be made of a different material, optionally less expensive, since second sections 2404 may extend along the entire length of the catheter body in this example. But as set forth herein, in alternative examples, the spines may not include second sections.

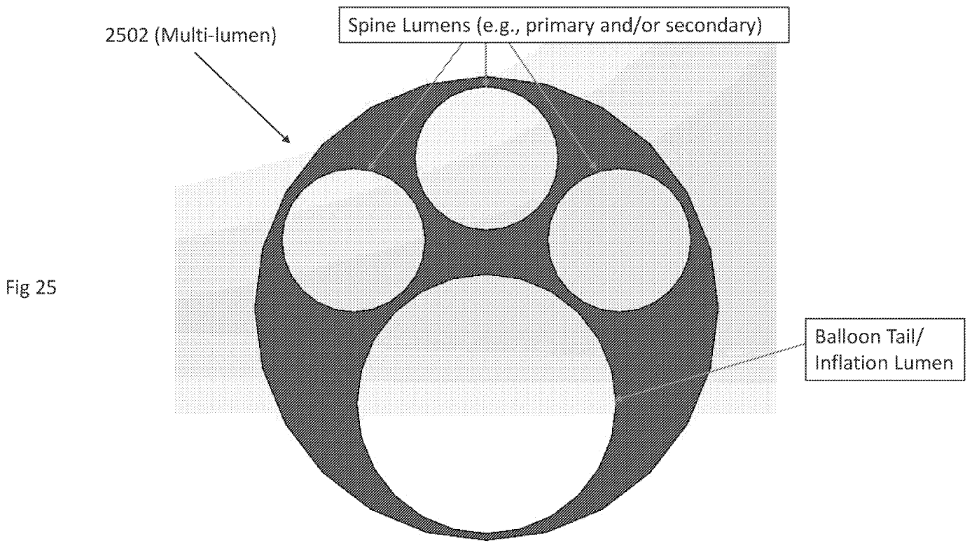

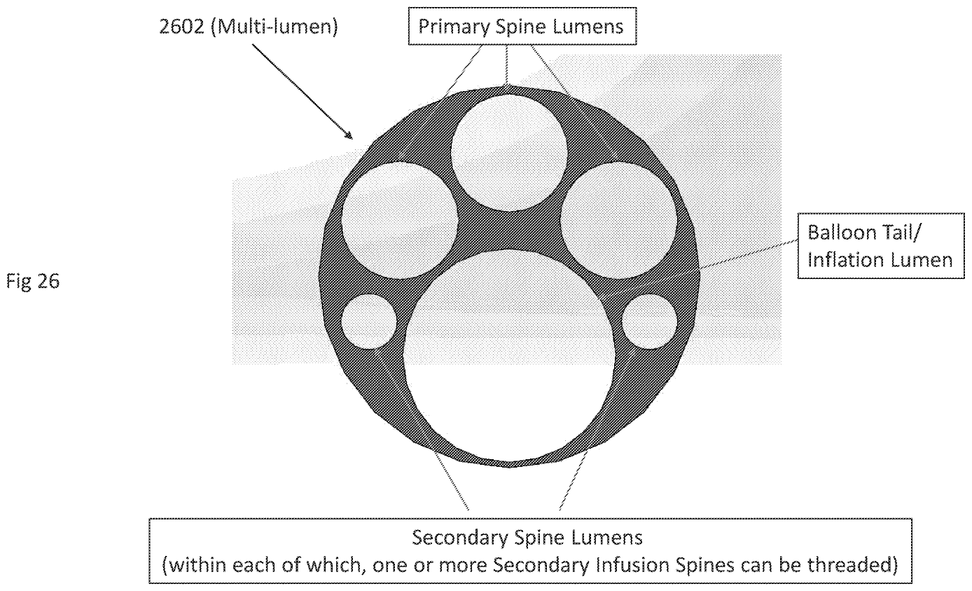

As shown in FIG. 24A, proximal ends of first sections 2402 are each disposed in a lumen of a catheter shaft, which may optionally be a spine lumen of a multi-lumen, cross sectional examples of which are shown in FIGS. 25 and 26. Each of the first sections 2402 is coupled to one of the second sections 2404 at a coupling location 2420, which in this example is within the catheter shaft, and optionally within a spine lumen of a multi-lumen. The first and second sections may be coupled together using a variety of techniques.

In alternatives to a multi-lumen, the catheter shaft may comprise a shaft that defines a single lumen, wherein in this alternative the spines are positioned within the same catheter shaft lumen.

A non-limiting exemplary advantage of a multi-lumen for the catheter shaft may be to provide structural support for the device along the length of the spines, such as if additional column strength is required or desired. A mere example of a first material for first sections 2402 is stainless steel. A mere example of a second material for second sections 2404 is a polyether ether ketone (“PEEK”).

An additional non-limiting exemplary advantage of a multi-lumen is to provide a plurality of conduits or lumens that may function as proximal extensions of spine sections 2402 if the spines do not include second sections 2404. With designs where spines do not include second sections 2404 (and thus do not extend all the way proximally through the catheter shaft), the spines (e.g., sections 2402) may be bonded to the ID of a spine lumen of the multi-lumen to provide a continuous conduit through which a movable needle assembly may be advanced.

Any of the multi-lumens herein may also be referred to as a multi-lumen tube, and they may be made from a variety of known materials with some degree of flexibility (e.g., polymeric materials) and made using a variety of known techniques.

The spines may be attached or secured to the catheter shaft, such as a multi-lumen, or they may not be secured thereto. In some instances, the spines may be free floating in the lumen(s) of the catheter shaft (e.g., multi-lumen). In some instances, they may have substantially the same OD as the ID of spine lumens of a multi-lumen.

FIG. 24B illustrates exemplary device 2400a (which may be similar or the same as 2400 in any respect), and shows optional guidewire lumen, a portion of which is within the balloon in this example, and which also extends proximally in the catheter shaft. In this context, the phrase “lumen” can refer to both the structure as well as the lumen that the structure defines.

FIG. 24B illustrates a proximal region of the balloon (referred to herein as a balloon “tail”) secured within a balloon tail lumen of the multi-lumen tube. Examples of balloon tail lumens are shown in the sectional views of the exemplary multi-lumens shown in FIGS. 25 and 26. The balloon tail lumens herein may also be referred to as balloon inflation lumens since the lumens serve as a conduit for balloon inflation fluid. The balloon tail is secured to the inner surface of the balloon tail lumen of the multi-lumen to provide a continuous conduit for balloon inflation.

Also, as shown in FIG. 24B, a guidewire lumen extends into the distal end of the balloon inflation lumen of the multi-lumen (and is within the balloon in this example). The guidewire lumen further extends along at least a portion of the length of the catheter shaft, and along the length of the balloon. The guidewire lumen may optionally extend all the way through the catheter shaft, but optionally it may not extend all the way through, such as with rapid-exchange designs where the guidewire exits the catheter shaft at some location distal to the proximal end of the catheter shaft. Whether the spines extend along the entire length of the catheter shaft or not, the device may comprise any of the movable needle assemblies herein, which may extend through primary spine lumens of a multi-lumen (and may also extend within a second section of the spine that is within the primary spine lumen).

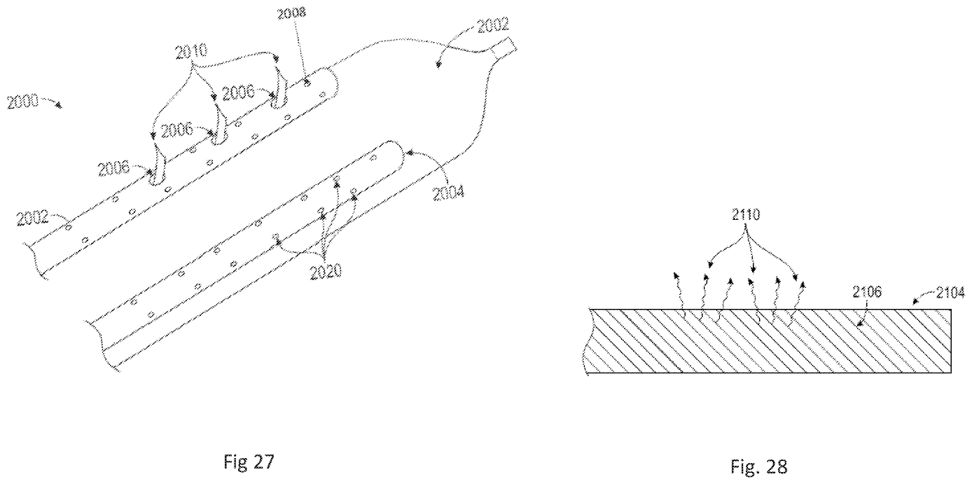

As shown in the sectional views of FIGS. 25 and 26, the optional multi-lumen tubes herein may comprise one or more secondary spine lumens in addition to one or more primary spine lumens and a balloon inflation lumen. The secondary spine lumens may be sized and configured to receive a secondary spine therein, examples of which are described in references incorporated by reference herein, including WO 2022/182598, published Sep. 1, 2022, and PCT/US2023/069886, filed Jul. 10, 2023, portions of which are provided below with reference to FIGS. 27, 28, 29A, 29B, 29B′ and 29C herein. Secondary spines can facilitate the delivery of a secondary agent therethrough, which is described below and in additional detail in the references incorporated by reference herein. The multi-lumen tubes herein may optionally comprise from one to ten spine lumens, optionally one to six, such as, without limitation, three spine lumens.

The sizes of the lumens of multi-lumen tubes may vary. A balloon inflation lumen may need to accommodate the size of a guidewire lumen and may need to be sized to provide sufficient space for delivery of the balloon inflation media, and therefore may be the largest lumen of the multi-lumen tube. Alternatively, the balloon inflation lumen may be a separate lumen within the multi-lumen than the guidewire lumen, and therefore may not be the largest lumen of the multi-lumen tube. The spine lumens may optionally be generally positioned more towards a first side of the multi-lumen than a second side, for example, to keep the overall outer diameter to a minimum in light of the size of the balloon inflation lumen, examples of which are shown in FIGS. 25 and 26.

The agent delivery devices herein may optionally include one or more secondary spines, examples of which are described herein, including in WO 2022/182598, published Sep. 1, 2022, and PCT/US2023/069886, filed Jul. 10, 2023, which are fully incorporated by reference herein, and wherein portions of which are provided below with reference to FIGS. 27, 28, 29A, 29B, 29B′ and 29C herein. A portion of any secondary spine herein may be disposed in a secondary spine lumen of a multi-lumen tube, examples of which are shown in FIGS. 25 and 26. While the multi-lumen in FIG. 26 includes two secondary spine lumens, multi-lumens herein may include one or more secondary spine lumens. Of the three spine lumens shown in FIG. 25, one or more may optionally be primary spine lumens, and one or more may optionally be secondary spine lumens, including any combination thereof.

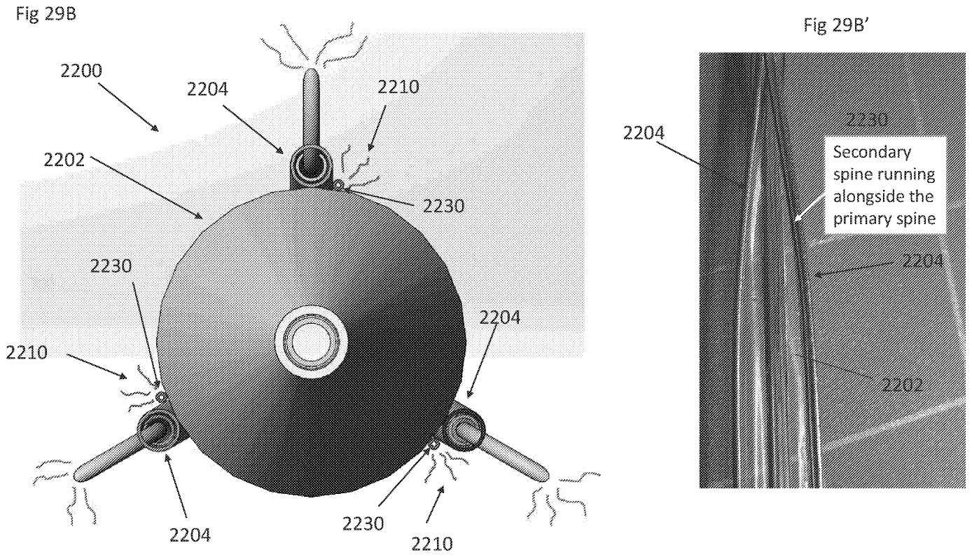

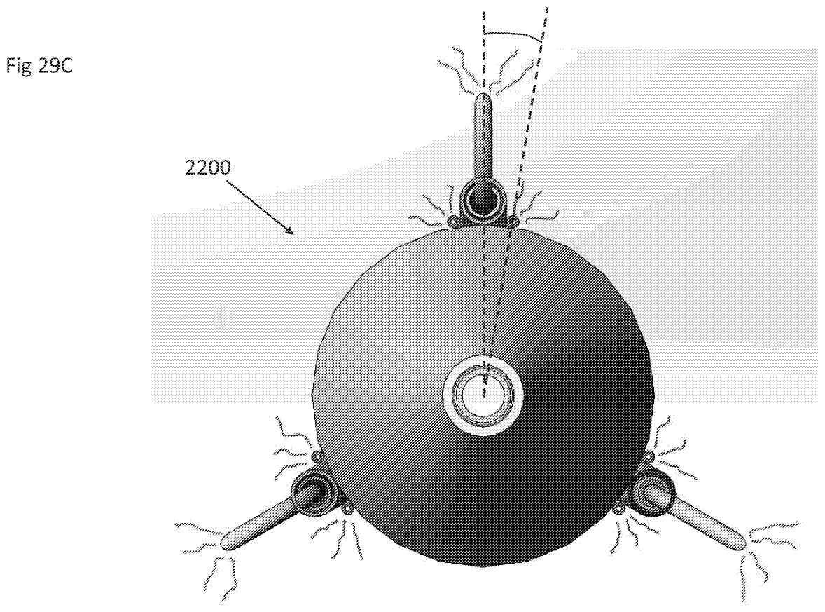

FIG. 26 is a sectional view of an exemplary multi-lumen tube that comprises a first set of primary spine lumens having a first diameter, and a second set of secondary spine lumens having a second diameter that is less than the first diameter. Secondary spine lumens herein may optionally be able to be made smaller than primary spine lumens due to smaller sizes of optional secondary spines, which can be smaller since they do not need to accommodate needle assemblies therein. For example, FIGS. 29A, 29B, 29B′, and 29C provide examples of secondary spines that are smaller in diameter than the primary spines. For at least this reason, it may thus be possible to include relatively smaller secondary spine lumens in the multi-lumen tube, as well as optionally position more than one secondary spine in any single secondary spine lumen (which may or may not be smaller in ID than primary spine lumens). For example, the agent delivery device in FIG. 29C comprises six secondary spines. If the device shown in FIG. 29C were combined with the multi-lumen tube in FIG. 26, there may be three secondary spines in each of the two secondary spine lumens, for example (or two secondary spines in a first secondary spine lumen, and four second spines in the second secondary spine, for example).

As previously described, in optional rapid-exchange designs, the guidewire lumen may terminate distal to the proximal end of the shaft. Terminating the guidewire lumen proximal to where the guidewire exists may be accomplished by closing off the lumen, for example thermally, or filling with another member such as a rod. It may therefore be beneficial to have a dedicated guidewire lumen that is distinct from the inflation lumen. As an alternative to that shown in FIG. 24B, any of the devices herein may include a multi-lumen, wherein one of the lumens may optionally serve as a guidewire lumen with a separate lumen serving as the balloon inflation lumen.

As described above generally with reference to FIGS. 24A, 24B, 25 and 26, any of the intravascular apparatus herein may also include one or more secondary spines, which are spines that include one or more secondary opening and do not include openings from which a needle is deployed.

Any of the secondary spines herein may be sized and configured to fit within one or more of the spine lumens shown in exemplary FIGS. 25 and 26. FIG. 27 illustrates a distal end of intravascular apparatus 2000 where balloon 2002 is inflated to a cylindrical configuration and primary spine 2002 and secondary spine 2004 are disposed about an outer cylindrical surface of the balloon 2002.

Primary spine 2002 includes primary openings 2006, each of which is associated with a deployable needle as is described herein. Secondary spine 2004 includes one or more secondary openings 2022, which may have any of the relative positions described herein. In some alternatives, the one or more secondary openings 2020 may include a discontinuity in the spine 2004, such as a laser cut gap in the spine, examples of which are described herein.

FIG. 27 also illustrates optional secondary openings 2008 in primary spine 2002, which may be any of the one or more secondary openings herein. When optional secondary openings 2008 are not included in spine 2002, apparatus 2000 is an example of an apparatus with at least one primary spine without any secondary openings, and with at least one secondary spine.

One aspect of the disclosure herein is a method of intravascular fluid delivery and treatment, comprising: advancing an intravascular apparatus to a target location within a vessel; inflating a balloon toward a cylindrical configuration to cause one or more primary spines of an expandable infusion scaffold to expand toward a vessel wall and be disposed about an outer cylindrical surface of the balloon when the balloon is inflated, wherein the one or more primary spines include a plurality of radial primary openings and optionally one or more secondary openings; moving a plurality of needles axially within the one or more primary spines and deploying the plurality of needles out of the radial primary openings such that tips of each of the plurality of needles pierce into the vessel wall; delivering a primary fluid agent out of the plurality of needles and into the vessel wall; and delivering a secondary fluid agent out of openings in at least one of the primary spine or a secondary spine. Delivering the secondary fluid agent may optionally include delivering the secondary agent through a primary spine lumen and out of one or more primary and/or secondary openings in the primary spine to expose the vessel wall to the secondary agent. Delivering the secondary fluid agent may optionally include delivering the secondary agent through a secondary spine lumen and out of one or more secondary openings in the secondary spine(s).

The delivering steps may comprise delivering the primary fluid agent deeper into the vessel wall than the secondary fluid agent, such as into the adventitia (and/or perivascular space) with the secondary fluid agent exposed to the surface of the vessel wall and optionally to the intima.

In some embodiments delivering the primary fluid agent can comprise delivering an anti-restenosis agent out of the plurality of needles and into the vessel wall.

In some embodiments, delivering the secondary fluid agent can comprise delivering an anti-recoil agent out of the one or more secondary openings to expose the vessel wall (for example, at least the intimal layer) to the anti-recoil agent.

In some embodiments, the primary fluid agent may be the same as the secondary fluid agent.

The secondary and primary agents may be delivered at the same time, or at different times. In some uses, there may be some overlap in their deliveries, even if the deliveries are initiated at different times. In some embodiments, the primary agent may comprise more than one agent (e.g., two or more different therapeutics), which may be delivered simultaneously (e.g., in combination) or separately at different times.

In some embodiments, delivering the secondary agent out of the one or more primary and/or secondary openings may be initiated before the plurality of needles are deployed from the radial primary openings. In some embodiments, delivering the secondary agent out of the one or more primary and/or secondary openings may be initiated at a time subsequent to when the plurality of needles are deployed from the radial primary openings.

In some embodiments, delivering the secondary agent out of the one or more primary and/or secondary openings is initiated at a time prior to delivering the primary fluid agent out of the plurality of needles.

In some embodiments, delivering the secondary agent out of the one or more primary and/or secondary openings occurs while the primary fluid agent is being delivered out of the plurality of needles.

In some embodiments, delivering the secondary agent out of the one or more primary and/or secondary openings is initiated at a time subsequent to delivering the primary fluid agent out of the plurality of needles.

In some embodiments, such as shown in FIG. 18 in WO 2022/182598 (incorporated by reference herein), delivering a secondary fluid agent through a primary spine lumen comprises delivering the secondary fluid agent between an inner surface of the primary spine and an outer surface of an axially moveable rail to which the plurality of needles is secured. The primary agent may be delivered through a lumen of the rail before it reaches the plurality of needles.

The primary and secondary fluid agents may optionally be disposed in first and second fluid sources outside of the patient when in use and in fluid communication with the primary and secondary openings. The devices herein may be placed into communication with one or more fluid agent sources prior to the procedure, and thus do not necessarily need to be in communication with the sources when packaged. This may allow one of several different agents and/or types of agents to be delivered with the fluid delivery devices herein.

The disclosure herein describes spines that may be optionally laser cut to impart flexibility along their lengths, which can increase flexibility for delivery. In some embodiments, the laser cuts in the spines may in fact constitute the one or more secondary openings in the spine, which allows the secondary agent to pass through the cut(s) and into the vessel wall. Laser cuts herein are examples of more generalized discontinuities in the wall of the spine, where the discontinuity is a secondary opening that facilitates weeping of the secondary agent therethrough.

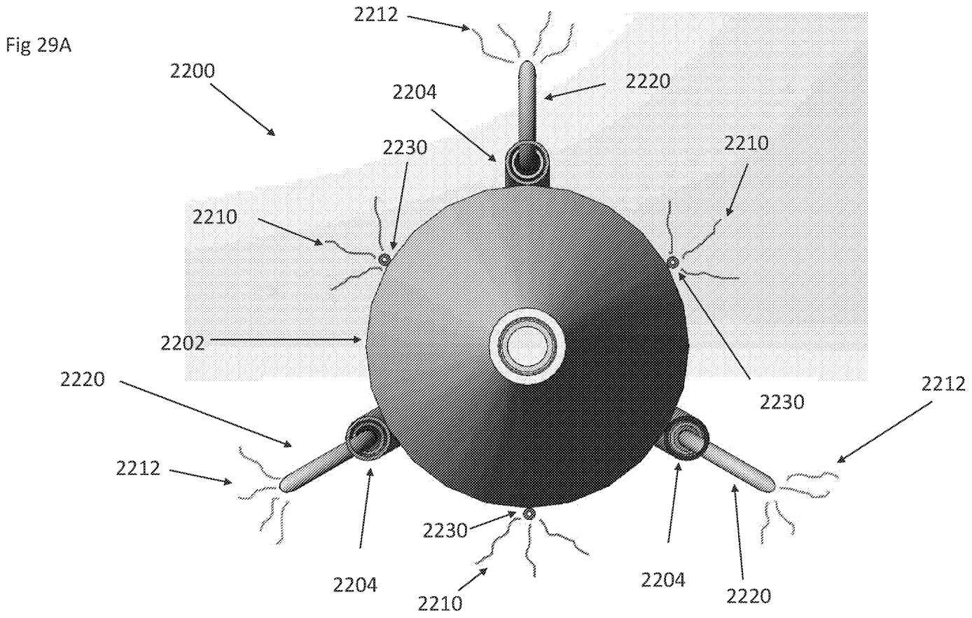

Both primary and optional secondary spines may include one or more discontinuities (e.g., one or more cuts therein) therein that are secondary openings. FIG. 28 is a side view of spine 2104 (which could be a primary or secondary spine) illustrating a secondary opening 2106 in the form of a laser cut helical pattern that facilitates delivery of secondary agent 2110 out of the spine 2104. In embodiments in which the spine has a laser cut pattern, part of the spine may be covered by a membrane to maintain fluid integrity, and the uncovered portion may act as the secondary opening. In these embodiments, the laser cut pattern may facilitate weeping of the secondary agent out of the secondary opening. In embodiments that include a laser cut pattern, a single, uninterrupted cut around the spine (e.g., in a helical configuration) may define a single secondary opening.

The needles may be adapted to be in communication with a first agent source outside the patient, and secondary openings may be adapted to be in communication with a second agent source outside the patient. The first and second sources may be the same sources, or they may be different sources. The different sources may contain therein the same agent or different agents.

FIGS. 29A, 29B and 29C illustrate end views of exemplary devices 2200 in expanded configurations with needles 2220 deployed from primary spines 2204. Any of the disclosure herein may optionally be incorporated into the devices 2200 in FIGS. 29A-29C (including methods of use thereof). The devices 2200 in FIGS. 29A-29C are similar to the device shown in FIG. 27, although the device in FIG. 27 includes a primary spine that also includes secondary openings, and the primary spines 2204 in FIGS. 29A-29C optionally do not include secondary openings (although they may be modified to include secondary openings).

In this example shown in FIG. 29A, secondary spines 2230 are equidistantly-spaced (or substantially equidistantly spaced) from the two circumferentially closest or nearest primary spines, as shown. Additionally, secondary spines 2230 optionally have smaller outer diameters than the primary spines, as shown. The secondary spines 2230 may be able to be smaller in the outermost dimension compared to the primary spines since they do not need to accommodate needles (and axially movable needle assemblies) therein. In some embodiments, the secondary spines may have an outer diameter (“OD”) that is not more than half the OD of the primary spines, for example, and optionally not more than 25% of the OD of the primary spines. The optionally smaller size of the secondary spines can help reduce the overall delivery profile of the device, compared to similar devices where the primary and secondary spines have the same OD. FIGS. 29A-29C also illustrate primary agent 2212 and secondary agent 2210 being delivered.

FIGS. 29B and 29B′ illustrate a variation on the device from FIG. 29A. Any unlabeled features of the device in FIGS. 29B and 29B′ may have the same reference numbers shown in FIG. 29A. The secondary spines 2230 in FIGS. 29B and 29B′ (only 1 is labeled in FIG. 29B′) are positioned closer or nearer to the primary spines 2204 compared to their position in the device in FIG. 29A. In this example, the secondary spines are adjacent and proximate the primary spines. In this context, the term “proximate” refers to being close to or near, and may optionally be in contact with, the primary spines. In this context, the secondary spines in FIGS. 29B and 29B′ that are “proximate” the primary spines are not equidistantly spaced from the circumferentially closest or nearest two primary spines, as shown. Placing secondary spines proximate the primary spines as shown may further help reduce the delivery profile of the device by placing the secondary spines in close proximity to the primary spines. The secondary spines may optionally comprise a flexible polymeric material, which may further help minimize the profile and/or trackability of the device.

For example only, the proximate secondary spines may be circumferentially offset between 0 and 45 degrees from the circumferentially closest primary spine (the angle measured circumferentially in the end view), or between 0 and 40 degrees, or between 0 and 35 degrees, or between 0 and 30 degrees, or between 0 and 25 degrees, or between 0 and 20 degrees, or between 0 and 15 degrees, or between 0 and 10 degrees, or between 0 and 5 degrees. For example, in FIG. 29B (and 29C) the proximate secondary spines are between 0 and 10 degrees from the circumferentially nearest primary spines, but this is understood to be exemplary and non-limiting.

FIG. 29C illustrates a variation on the device 2200 in FIGS. 29B and 29B′, in which the device includes first and second secondary spines that are proximate to each of the primary spines, as shown. Any unlabeled features of the device in FIG. 29C may have the same reference numbers shown in the devices in FIGS. 29A-29B′. Any of the disclosure from FIGS. 29A-29B′ may be incorporated into the device in FIG. 29C. First and second secondary spines are proximate to and on opposite sides of each of a primary spine in this example, as shown. Having a plurality of secondary spines about each primary spine may help expose more of the vessel wall to the secondary fluid agent, without significantly increasing the delivery profile. In this example two of the secondary spines are the same angle from the circumferentially nearest primary spine, but in variations of FIG. 29C the two secondary spines may be at different angles from the nearest primary spine (e.g., one at 10 degree and the other at 20 degrees).

The end view of FIG. 29C illustrates an angle between a primary spine and a secondary spine. As shown, the angle is defined by two dashed lines, one extending from a device long axis through a longitudinal axis of a primary spine and the other extending from the device long axis through a longitudinal axis of a secondary spine.

Any of the secondary spines herein may be coupled (directly or indirectly) to at least some portion of the balloon and/or at least some portion of primary spines that are in close proximity to the secondary spines.

1. A method of denervating renal nerves, comprising:
advancing an intravascular apparatus into a renal artery, the apparatus including an expandable cylindrical balloon and a plurality of spines;
inflating the balloon towards a cylindrical configuration to cause the plurality of spines to move radially towards the renal artery wall, each of the spines including a proximal radial opening therethrough and a distal radial opening therethrough;
deploying a proximal set of needles from the proximal radial openings in the plurality of spines and into tissue at least one of within or surrounding the renal artery wall, wherein a single needle from the proximal set of needles is deployed from each of the proximal radial openings;
deploying a distal set of needles from the distal radial openings in the plurality of spines and into tissue at last one of within or surrounding the renal artery wall, wherein a single needle from the distal set of needles is deployed from each of the distal radial openings,
wherein the distal set of needles is distally spaced from the proximal set of needles, and wherein the proximal set of needles extend further radially outward from the spines than the distal set of needles when deployed; and
at a time subsequent to the deploying steps, delivering an ablative agent from the proximal set of needles to create a proximal ablation zone and from the distal set of needles to create a distal ablation zone.

2. The method of claim 1, wherein the proximal ablation zone overlaps with the distal ablation zone.

3. The method of claim 1, wherein deploying the proximal set of needles occurs while deploying the distal set of needles.

4. The method of claim 3, wherein deploying the proximal set of needles and deploying the distal set of needles comprises axially moving a rail member within and relative to each spine, each axially movable rail member coupled to one of the distal set of needles and one of the proximal set of needles.

5. The method of claim 1, wherein deploying the proximal set of needles from the proximal radial openings comprises deploying the proximal set of needles to each extend from the respective spine a distance from 1.5 mm to 6 mm.

6. The method of claim 5, wherein the distance is 2 mm to 4 mm.

7. The method of claim 5, wherein deploying the distal set of needles from the distal radial openings comprises deploying the distal set of needles to each extend from the respective spine a distance from 0.5 mm to 4 mm.

8. The method of claim 7, wherein the distal needles each extend from the respective spine a distance from 1 mm to 3 mm.

9. The method of claim 1, wherein deploying the distal set of needles from the distal radial openings comprises deploying the distal set of needles to each extend from the respective spine a distance from 0.5 mm to 4 mm.

10. The method of claim 9, wherein the distance is 1 mm to 3 mm.

11. The method of claim 1, wherein deploying the proximal set of needles comprises deploying the proximal set of needles so that proximal needles are all axially aligned in a row.

12. The method of claim 11, wherein deploying the distal set of needles comprises deploying the distal set of needles so that distal needles are all axially aligned in a row.

13. The method of claim 1, wherein deploying the distal set of needles comprises deploying the distal set of needles so that the distal needles are all axially aligned in a row.

14. The method of claim 1, wherein the plurality of spines are secured to an outer surface of the balloon.

15. The method of claim 1, wherein each of the proximal set of needles is axially aligned in a proximal row when deployed, and wherein each of the distal set of needles is axially aligned in a distal row when deployed, and wherein the apparatus does not include more rows than the proximal row and the distal row.

16. The method of claim 1, wherein creating the proximal ablation zone and creating the distal ablation zone comprises creating a total ablation region that has a generally tapered configuration that tapers downward in the proximal to distal direction.

17.-38. (canceled)
